(19) United States
(12) Patent Application Publication
(10) Pub. No.: US 2022/0401423 A1
(43) Pub. Date: Dec. 22, 2022

(54) METHODS FOR TREATING INFLAMMATORY BOWEL DISEASE

(71) Applicant: Angion Biomedica Corp., Uniondale (US)

(72) Inventor: Swarnalatha Paka, Elmont, NY (US)

(21) Appl. No.: 17/778,280

(22) PCT Filed: Nov. 19, 2020
(86) PCT No.: PCT/US2020/061322

§ 371 Date: May 19, 2022

Related U.S. Application Data
(60) Provisional application No. 62/938,248, filed on Nov. 20, 2019.

(51) Int. Cl. A61K 31/437; A61P 1/04

(52) U.S. Cl. CPC A61K 31/437; A61P 1/04

(57) ABSTRACT

The present disclosure provides methods for treating inflammatory bowel disease including Crohn's disease and ulcerative colitis by administering a compound of Formula (I) and pharmaceutically acceptable derivatives thereof, as described generally and in classes and subclasses herein, and additionally provides pharmaceutical compositions thereof.

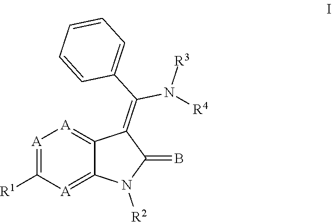

### RELATED APPLICATIONS

This application claims priority to U.S. Provisional Patent Appl. No. 62/938,248, filed Nov. 20, 2019, the entire contents of which are hereby incorporated by reference.

### BACKGROUND

Inflammatory bowel disease (IBD) is an inflammatory condition that comprises both ulcerative colitis (UC) and Crohn's disease (CD). While UC affects the entire colon, CD typically affects the ileum but can occur any part of GI tract. IBD can manifest as acute or chronic colitis, characterized by recurrent intestinal inflammation accompanied by diarrhea and abdominal pain. Recurring bouts of inflammation can lead to tissue remodeling and is a serious presentation in IBD and is a major cause of morbidity, often requiring hospitalization and surgical intervention.

The incidence of IBD is increasing worldwide and is an expanding global health problem. An estimated 2.5-3 million people in Europe are affected by IBD. According to the Centers for Disease Control and Prevention (CDC), 3.1 million adults in the United States were diagnosed with IBD (i.e. CD or UC) in 2015, a substantial increase from the ˜1.4 million adults diagnosed per 2008 reports. IBD accounts for ˜1,300,000 physician visits and ˜92,000 hospitalizations each year in the United States. Of these, 75% patients diagnosed with CD and 25% patients diagnosed with UC require surgery. Risk factors associated with IBD include environmental, genetic and immunologic factors.

IBD is a major cause of morbidity in patients and is a major consumer of the health care budget. A European study estimated that direct healthcare costs for IBD in Europe are ˜5 billion per year. In 2008, CDC reports indicate that direct treatment costs for IBD in the United States were estimated to be ˜$6.3 billion and indirect costs were estimated to be an additional $5.5 billion.

IBD is an autoimmune disease characterized by excessive activation of the adaptive immune response. Various factors, including genetic factors, alter the intestinal flora and trigger an inflammatory reaction, which activates T cells, B cells, mast cells, macrophages and microglia, smooth muscle cells and fibroblasts in the colon, thereby inducing mucosal disruption. Epithelial and endothelial damage release chemotactic factors promoting recruitment and activation of inflammatory cells and release of various cytokines.

Intestinal fibrosis, commonly defined as an excessive deposition of extracellular matrix (ECM) resulting from chronic inflammation and impairment of intestinal wound healing, represents a serious complication of IBD and has important clinical implications. This is true for both UC and CD. In UC, the involvement of the mucosal and submucosal layers causes a thickening of the muscularis mucosae with accumulation of ECM that may contribute to shortening or stiffening of the colon, whereas in CD the transmural nature of the inflammatory process is followed by bowel wall thickening, and eventually formation of stricture and stenosis. More than a third of patients with CD develop a distinct fibro-stenosing phenotype, manifested by progressive narrowing of the intestinal lumen and potential obstruction. Together with fistulae, intestinal stenosis represents the main indication for surgery in CD, whereas in UC, indication of surgery due to bowel stenosis is more sporadic. Up to 80% of all patients suffering from CD undergo surgery at least once during the course of their disease. In approximately half of these patients, stricture formation and obstruction secondary to bowel wall fibrosis are the main reason for surgery, denoting that excessive scar tissue formation is underlying the need for an operation in approximately one-third of all CD patients. Recurrence of disease at the site of anastomosis is common, and recurrent stricture formation may also occur. Progressive intestinal fibrosis leads to circumferential bowel wall thickening, impaired peristalsis and bowel obstruction.

### SUMMARY

The present disclosure provides certain technologies for improved treatment of inflammatory bowel disease (IBD), including Crohn's disease and ulcerative colitis. In some embodiments, the present disclosure provides methods of treating IBD using compounds described herein.

In some embodiments, the present disclosure provides methods of treating (e.g., lessening the severity of such as by delaying onset and/or reducing degree and/or frequency of one or more features of), IBD, which methods may comprise administering a compound of Formula (I):

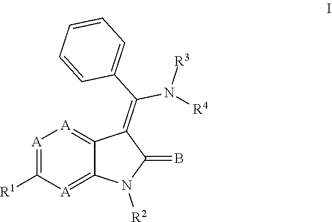

or a pharmaceutically acceptable salt thereof or a prodrug thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷;
R⁵ is a lower alkyl group;
R⁶ and R⁷ are independently hydrogen, alkyl, cycloalkyl, heterocycle, cycloalkylalkyl, alkylcycloalkylalkyl, heterocycloalkyl or alkylheterocycloalkyl, which may be optionally substituted with alkyl, OR⁸, COOR⁸, NR⁸R⁹, or NHCOR⁸;
R⁸ and R⁹ are independently H or a lower alkyl group;
A is N or CH, wherein one A is nitrogen; and
B is O or S.

In some embodiments, the methods described herein comprise administering compounds of Formula (II):

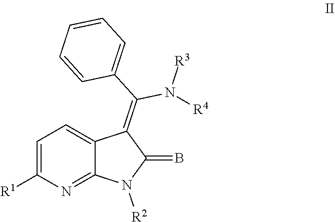

or a pharmaceutically acceptable salt thereof or a prodrug thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷;
R⁵ is a lower alkyl group;
R⁶ and R⁷ are independently hydrogen, alkyl, cycloalkyl, heterocycle, cycloalkylalkyl, alkylcycloalkylalkyl, heterocycloalkyl or alkylheterocycloalkyl, which may be optionally substituted with alkyl, OR⁸, COOR⁸, NR⁸R⁹, or NHCOR⁸;
R⁸ and R⁹ are independently H or a lower alkyl group; and
B is O or S.

In some embodiments, the methods described herein comprise administering compounds of Formula (III):

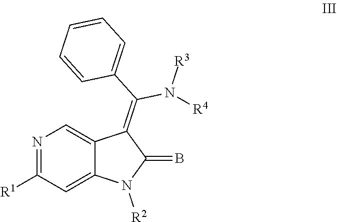

or a pharmaceutically acceptable salt thereof or a prodrug thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷;
R⁵ is a lower alkyl group;
R⁶ and R⁷ are independently hydrogen, alkyl, cycloalkyl, heterocycle, cycloalkylalkyl, alkylcycloalkylalkyl, heterocycloalkyl or alkylheterocycloalkyl, which may be optionally substituted with alkyl, OR⁸, COOR⁸, NR⁸R⁹, or NHCOR⁸;
R⁸ and R⁹ are independently H or a lower alkyl group; and
B is O or S.

In some embodiments, the methods described herein comprise administering compounds of Formula (IV):

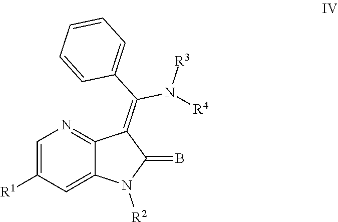

or a pharmaceutically acceptable salt thereof or a prodrug thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷;
R⁵ is lower alkyl group;
R⁶ and R⁷ are independently hydrogen, alkyl, cycloalkyl, heterocycle, cycloalkylalkyl, alkylcycloalkylalkyl, heterocycloalkyl or alkylheterocycloalkyl, which may be optionally substituted with alkyl, OR⁸, COOR⁸, NR⁸R⁹, or NHCOR⁸;
R⁸ and R⁹ are independently H or a lower alkyl group; and
B is O or S.

In some embodiments, provided methods comprise administering compounds described herein via any suitable route of administration. In some embodiments, provided compounds are delivered by any route that provides benefit to the disease. In some embodiments, provided compounds are administered orally, rectally, parenterally, intraperitoneally, or subcutaneously, by way of non-limiting examples.

In some embodiments, the present disclosure also provides pharmaceutical compositions comprising a compound described herein and, optionally, a pharmaceutically acceptable carrier, excipient or diluent. Such pharmaceutical compositions are useful, e.g., in methods provided herein.

In some embodiments, the present disclosure encompasses the recognition that delivery of provided compounds to the colon may be desirable for treating IBD. Accordingly, in some embodiments, the present disclosure contemplates compositions with release characteristics particularly suited for treatment of IBD (e.g., through delivery of a compound to the colon).

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1A, FIG. 1B, FIG. 1C, and FIG. 1D show pre-randomization (pre-Rx) body weight, colon weight, colon length, and colon gross damage score, respectively, of mice in the acetic acid (AA) induced colitis model.

FIG. 2A, FIG. 2B, FIG. 2C, FIG. 2D, and FIG. 2E show that treatment with a test compound (TC; Compound 1) significantly decreased AA-induced colitis in a mouse model, as evidenced by colon length, colon gross damage score, histopathological damage score, Alcian blue staining, and hematoxylin and eosin (H&E) staining, respectively.

FIG. 3 shows that treatment with a test compound increased survival in the 2,4,6-trinitrobenzenesulfonic acid (TNBS) induced colitis mortality model.

FIG. 4A, FIG. 4B, FIG. 4C, and FIG. 4D show body weight, colon length, and colon damage score of mice in the TNBS-induced colitis model prior to randomization.

FIG. 5 demonstrates the effect of increasing doses of a test compound on body weight of mice in the TNBS-induced colitis model.

FIG. 5, FIG. 6, FIG. 7, FIG. 8, and FIG. 9 show that animals in the TNBS-induced colitis model recover from IBD symptoms when treated with a test compound, namely body weight (FIG. 5), colon length (FIG. 6), colon weight (FIG. 7), colon length (FIG. 8) and disease severity index (FIG. 9).

FIG. 10 depicts histology of colons of animals in the TNBS-induced colitis model. The colon histopathological scores are shown in FIG. 11 and FIG. 12.

FIG. 13 depicts Alcian blue staining of the colons of animals in the TNBS-induced colitis model. The Alcian blue staining (% area) for goblet cells is shown in FIG. 14 and for crypt cells in FIG. 15.

FIG. 16 depicts myeloperoxidase (MPO) staining of the colons of animals in the TNBS-induced colitis model. The MPO staining percent covered area is shown in FIG. 17 and FIG. 18.

FIG. 19 depicts F4/80 staining of the colons of animals in the TNBS-induced colitis model. The MPO staining percent covered area is shown in FIG. 20 and FIG. 21.

FIG. 22A, FIG. 22B, FIG. 22C, FIG. 22D, FIG. 22E, and FIG. 22F demonstrate that treatment with a test compound significantly restored colon length (FIG. 22A and FIG. 22B), decreased colon damage score (FIG. 22C), and decreased hydroxyproline (HYP) levels (FIG. 22D) in the DSS IBD mouse model. The colon histopathologic score and histology of the colons of animals in the DSS IBD model is shown in FIG. 22E and FIG. 22F, respectively.

FIG. 23 shows the antifibrotic activity of a test compound, as evidenced by reduced collagen levels upon treatment.

FIG. 24A, FIG. 24B, and FIG. 24C show body weight, colon length, and colon damage score of mice in the TNBS-induced colitis model prior to randomization.

FIG. 25A, FIG. 25B, FIG. 25C, FIG. 25D, FIG. 25E, and FIG. 25F show that animals in the TNBS-induced colitis model recover from IBD symptoms when treated with a test compound, namely colon length (FIG. 25A and FIG. 25B), colon gross damage score (FIG. 25C), Alcian blue staining of goblet cells (FIG. 25D and FIG. 25E), and colon histopathological score (FIG. 25F).

FIG. 26 shows treatment with a test compound significantly blocked TNBS-induced increase in serum cytokines in an TNBS-induced colitis mouse model.

### DEFINITIONS

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented. For example, in some embodiments, “treatment” describes improvement, remission, benefit, of any of the one or more physical and pathophysiological signs of various forms of IBD.

The terms “subject,” “individual,” and “patient” are used interchangeably herein, and refer to an animal, for example a human. In some embodiments, a subject is a human. The human can be any human of any age. In some embodiments, the human is an adult. In some embodiments, the human is a child. The human can be male, female, pregnant, middle-aged, adolescent, and/or elderly. In some embodiments, a subject is a non-human animal, such as, e.g., mammals, such as non-human primates, (particularly higher primates), sheep, dogs, rodents, (e.g. mouse or rat), guinea pigs, goats, pigs, cats, rabbits, cows, horses and non-mammals such as reptiles, amphibians, chickens, and turkeys. The compositions described herein can be used to treat any suitable mammal, including primates such as monkeys and humans, horses, cows, cats, dogs, rabbits, and rodents such as rats and mice. In some embodiments, the subject is a non-human primate. In some embodiments, the subject is murine, which in one embodiment is a mouse, and, in another embodiment is a rat. In some embodiments, the subject is canine, feline, bovine, equine, laprine or porcine. In some embodiments, the subject is mammalian. In some embodiments, a subject is at risk of (e.g., susceptible to), e.g., at elevated risk relative to an appropriate control individual or population thereof, a disease disorder or condition. In some embodiments, a subject displays one or more symptoms or characteristics of a disease, disorder or condition. In some embodiments, a subject does not display any symptom or characteristic of a disease, disorder or condition. In some embodiments, a subject is an individual to whom diagnosis and/or therapy and/or prophylaxis is and/or has been administered.

As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

As described herein, it will be appreciated that provided compounds, e.g., useful for the methods as described herein, may be substituted with any number of substituents or functional moieties, as valency permits. In general, the term “substituted” whether preceded by the term “optionally” or not, refers to the replacement of one or more hydrogen radicals in a given structure with the radical of a specified substituent. When more than one position in any given structure may be substituted with more than one substituent selected from a specified group, the substituent may be either the same or different at every position. As used herein, the term “substituted” is contemplated to include substitution with any permissible substituent. In some embodiments, permissible substituents include acyclic and cyclic, branched and unbranched, carbocyclic and heterocyclic, aromatic and non-aromatic, carbon and heteroatom substituents of organic compounds. For purposes of this disclosure, heteroatoms such as nitrogen may have hydrogen substituents and/or any permissible substituents of organic compounds described herein which satisfy the valencies of the heteroatoms. Combinations of substituents and variables envisioned by this disclosure are preferably those that result in the formation of stable compounds, e.g., useful in the treatment and prevention, for example of disorders, as described generally above. Groups described as being “substituted” preferably have between 1 and 4 substituents, more preferably 1 or 2 substituents. Groups described as being “optionally substituted” may be unsubstituted or “substituted” as described above.

Examples of substituents include, but are not limited to aliphatic; heteroaliphatic; alicyclic; heterocyclic; aromatic, heteroaromatic; aryl; heteroaryl; alkylaryl; aralkyl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; or -GR^{G1} wherein G is —O—, —S—, —NR^{G2}—, —C(O), —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, alicyclic, heterocyclic, aromatic, heteroaromatic, aryl, heteroaryl, alkylaryl, or alkylheteroaryl moiety. In some embodiments, each occurrence of R^{G1}, R^{G2} and R^{G3} independently includes, but is not limited to, hydrogen, halogen, or an aliphatic, heteroaliphatic, alicyclic, heterocyclic, aromatic, heteroaromatic, aryl, heteroaryl, alkylaryl, or alkylheteroaryl moiety. Additional examples of generally applicable substituents are illustrated by the specific embodiments shown in the Examples that are described herein.

The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched) or branched aliphatic hydrocarbons as defined by IUPAC, which are optionally substituted with one or more functional groups, as described herein. As defined herein, “aliphatic” is intended to include optionally substituted alkyl, alkenyl and alkynyl moieties. Thus, as used herein, the term “alkyl” includes straight and branched alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (substituted, unsubstituted, branched or unbranched) having about 1-6 carbon atoms. In some instances aliphatic can include alicyclic or cycloalkyl, including unsaturations therein.

In some embodiments, the alkyl, alkenyl and alkynyl groups employed in the disclosure contain 1-20; 2-20; 3-20; 4-20; 5-20; 6-20; 7-20 or 8-20 aliphatic carbon atoms. In some embodiments, the alkyl, alkenyl, and alkynyl groups employed in the disclosure contain 1-10; 2-10; 3-10; 4-10; 5-10; 6-10; 7-10 or 8-10 aliphatic carbon atoms. In some embodiments, the alkyl, alkenyl, and alkynyl groups employed in the disclosure contain 1-8; 2-8; 3-8; 4-8; 5-8; 6-20 or 7-8 aliphatic carbon atoms. In some embodiments, the alkyl, alkenyl, and alkynyl groups employed in the disclosure contain 1-6; 2-6; 3-6; 4-6 or 5-6 aliphatic carbon atoms. In some embodiments, the alkyl, alkenyl, and alkynyl groups employed in the disclosure contain 1-4; 2-4 or 3-4 carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “alicyclic”, as used herein, refers to moieties that combine the properties of aliphatic and cyclic compounds and include but are not limited to cyclic, or polycyclic aliphatic hydrocarbons and bridged cycloalkyl compounds, which are optionally substituted with one or more functional groups. As will be appreciated by one of ordinary skill in the art, “alicyclic” is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH₂-cyclopropyl, cyclobutyl, —CH₂-cyclobutyl, cyclopentyl, —CH₂-cyclopentyl, cyclohexyl, —CH₂-cyclohexyl, cyclohexenylethyl, cyclohexanylethyl, norbomyl moieties and the like, which again, may bear one or more substituents.

The term “cycloalkyl”, as used herein, refers to cyclic alkyl groups, specifically to groups having three to seven, preferably three to ten carbon atoms. Suitable cycloalkyls include, but are not limited to cyclopropyl, cyclobutyl, cyclopentyl, cyclohexyl, cycloheptyl and the like, which may optionally be substituted. An analogous convention applies to other generic terms such as “cycloalkenyl”, “cycloalkynyl” and the like. Additional examples of generally applicable substituents are illustrated by the specific embodiments shown in the Examples that are described herein.

The term “heteroaliphatic”, as used herein, refers to aliphatic moieties in which one or more carbon atoms in the main chain have been replaced with a heteroatom. Thus, a heteroaliphatic group refers to an aliphatic chain which contains one or more oxygen, sulfur, nitrogen, phosphorus or silicon atoms in place of carbon atoms in the aliphatic main chain. Heteroaliphatic moieties may be branched or linear unbranched. In certain embodiments, heteroaliphatic moieties are substituted as described herein. Additional examples of generally applicable substituents are illustrated by the specific embodiments shown in the Examples that are described herein.

The terms “heteroalicyclic”, “heterocycloalkyl” or “heterocyclic”, as used herein, refer to moieties which combine the properties of heteroaliphatic and cyclic moieties and include but are not limited to saturated and unsaturated mono- or polycyclic ring systems having 5-16 atoms wherein at least one ring atom is a heteroatom selected from O, S and N (wherein the nitrogen and sulfur heteroatoms may be optionally be oxidized), wherein the ring systems are optionally substituted with one or more functional groups, as defined herein. In certain embodiments, the term “heterocyclic” refers to a non-aromatic 5-, 6- or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may optionally be oxidized, (iii) the nitrogen heteroatom may optionally be quaternized, and (iv) any of the above heterocyclic rings may be fused to an aryl or heteroaryl ring. Representative heterocycles include, but are not limited to, pyrrolidinyl, pyrazolinyl, pyrazolidinyl imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted as described herein. Additional examples or generally applicable substituents are illustrated by the specific embodiments shown in the Examples, which are described herein.

Additionally, it will be appreciated that any of the alicyclic or heterocyclic moieties described above and herein may comprise an aryl or heteroaryl moiety fused thereto.

In general, the term “aromatic moiety”, as used herein, refers to a stable mono- or polycyclic, unsaturated moiety having preferably 3-14 carbon atoms (e.g., 6-14 carbon atoms), each of which may be substituted or unsubstituted. In certain embodiments, the term “aromatic moiety” refers to a moiety comprising at least one planar ring having p-orbitals perpendicular to the plane of the ring at each ring atom and satisfying the Huckel rule where the number of pi electrons in the ring is (4n+2) wherein n is an integer. A mono- or polycyclic, unsaturated moiety that does not satisfy one or all of these criteria for aromaticity is defined herein as “non-aromatic”, and is encompassed by the term “alicyclic”. Examples of aromatic moieties include, but are not limited to, phenyl, indanyl, indenyl, naphthyl, phenanthryl and anthracyl.

In general, the term “heteroaromatic moiety”, as used herein, refers to stable substituted or unsubstituted unsaturated mono-heterocyclic or polyheterocyclic moieties having preferably 3-14 ring members (e.g., 5-10 ring members) and 1-4 heteroatoms selected from nitrogen, oxygen, and sulfur, comprising at least one ring having p-orbitals perpendicular to the plane of the ring at each ring atom, and satisfying the Huckel rule where the number of pi electrons in the ring is (4n+2) wherein n is an integer. Examples of heteroaromatic moieties include, but are not limited to, pyridyl, quinolinyl, dihydroquinolinyl, isoquinolinyl, quinazolinyl, dihydroquinazolyl, and tetrahydroquinazolyl.

It will also be appreciated that aromatic and heteroaromatic moieties, as defined herein, may be attached via an aliphatic (e.g., alkyl) or heteroaliphatic (e.g., heteroalkyl) moiety and thus also include moieties such as -(aliphatic)aromatic, -(heteroaliphatic)aromatic, -(aliphatic)heteroaromatic, -(heteroaliphatic)heteroaromatic, -(alkyl)aromatic, -(heteroalkyl)aromatic, -(alkyl)heteroaromatic, and -(heteroalkyl)heteroaromatic moieties. Thus, as used herein, the phrases “aromatic or heteroaromatic moieties” and “aromatic, heteroaromatic, -(alkyl)aromatic, -(heteroalkyl)aromatic, -(heteroalkyl)heteroaromatic, and -(heteroalkyl)heteroaromatic” are interchangeable. In some instances, corresponding moieties may be referred to synonymously as aralkyl, heteroaralkyl and the like. Substituents include, but are not limited to, any of the previously mentioned substituents, i.e., the substituents recited for aliphatic moieties, or for other moieties as disclosed herein, resulting in the formation of a stable compound.

In general, the term “aryl” refers to aromatic moieties, as described above, excluding those attached via an aliphatic (e.g., alkyl) or heteroaliphatic (e.g., heteroalkyl) moiety. In certain embodiments of the present disclosure, “aryl” refers to a mono- or bicyclic carbocyclic ring system having one or two rings satisfying the Huckel rule for aromaticity, including, but not limited to, phenyl, naphthyl, tetrahydronaphthyl, indanyl, indenyl and the like.

Similarly, the term “heteroaryl” refers to heteroaromatic moieties, as described above, excluding those attached via an aliphatic (e.g., alkyl) or heteroaliphatic (e.g., heteroalkyl) moiety. In certain embodiments of the present disclosure, the term “heteroaryl”, as used herein, refers to a cyclic unsaturated radical having from about five to about ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

As defined herein, “aryl” and “heteroaryl” groups (including bicyclic aryl groups) can be unsubstituted or substituted, as described herein. Additionally, it will be appreciated, that any two adjacent groups taken together may form a 4, 5, 6, or 7-membered substituted or unsubstituted alicyclic or heterocyclic moiety. Additional examples of generally applicable substituents are illustrated by the specific embodiments shown in the Examples that are described herein.

The term “alkoxy” or “alkyloxy”, as used herein refers to a saturated (i.e., O-alkyl) or unsaturated (i.e., O-alkenyl and O-alkynyl) group attached to the parent molecular moiety through an oxygen atom. In some embodiments, the alkyl group contains 1-20; 2-20; 3-20; 4-20; 5-20; 6-20; 7-20 or 8-20 aliphatic carbon atoms. In some embodiments, the alkyl group contains 1-10; 2-10; 3-10; 4-10; 5-10; 6-10; 7-10 or 8-10 aliphatic carbon atoms. In some embodiments, the alkyl, alkenyl, and alkynyl groups employed in the invention contain 1-8; 2-8; 3-8; 4-8; 5-8; 6-20 or 7-8 aliphatic carbon atoms. In some embodiments, the alkyl group contains 1-6; 2-6; 3-6; 4-6 or 5-6 aliphatic carbon atoms. In some embodiments, the alkyl group contains 1-4; 2-4 or 3-4 aliphatic carbon atoms. Examples of alkoxy, include but are not limited to, methoxy, ethoxy, propoxy, isopropoxy, n-butoxy, i-butoxy, sec-butoxy, tert-butoxy, neopentoxy, n-hexoxy and the like.

The term “alkylthio” as used herein refers to a saturated (i.e., S-alkyl) or unsaturated (i.e., S-alkenyl and S-alkynyl) group attached to the parent molecular moiety through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylthio include, but are not limited to, methylthio, ethylthio, propylthio, isopropylthio, n-butylthio, and the like.

The terms “halo” and “halogen” as used herein refer to an atom selected from fluorine, chlorine, bromine and iodine.

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester, which is cleaved in vivo to yield a compound of interest. Another example is an N-methyl derivative of a compound, which is susceptible to oxidative metabolism resulting in N-demethylation. Pro-drugs of a variety of compounds, and materials and methods for derivatizing the parent compounds to create the pro-drugs, are known and may be adapted to the present disclosure. Certain exemplary pharmaceutical compositions and pharmaceutically acceptable derivatives will be discussed in more detail herein below.

The term “tautomerization” refers to the phenomenon wherein two or more structural isomers of a compound readily interconvert. Commonly, tautomerization occurs via a proton shift from one atom of a molecule another. See, Jerry March, Advanced Organic Chemistry: Reactions, Mechanisms and Structures, Fourth Edition, John Wiley & Sons, pages 69-74 (1992). The term “tautomer” as used herein, refers to the structural isomers that readily interconvert (e.g., compounds produced by the proton shift).

As used herein, the term “isolated” when applied to the compounds of the present disclosure, refers to such compounds that are (i) separated from at least some components with which they are associated in nature or when they are made and/or (ii) produced, prepared or manufactured by the hand of man.

### DETAILED DESCRIPTION

The present subject matter may be understood more readily by reference to the following detailed description which forms a part of this disclosure. It is to be understood that this disclosure is not limited to the specific products, methods, conditions or parameters described and/or shown herein, and that the terminology used herein is for the purpose of describing particular embodiments by way of example only and is not intended to be limiting of the claimed invention.

Inflammatory bowel disease (IBD) including Crohn's disease and ulcerative colitis afflicts a large population and has significant adverse consequences on the quality, as well as duration, of life. The compounds described herein have been found to be effective in models of IBD that resemble human disease and thus demonstrate utility for the treatment of IBD in humans. As described in the examples below, using a TNBS model, an acetic acid model and a DSS model, an exemplary compound provided numerous benefits to animals in IBD models, including but not limited to improvements in body weight, colon length, colon weight, gross morphological disease severity index, colon histopathological score, crypt architecture and inflammatory markers myeloperoxidase and F4/80.

Inflammatory bowel disease (IBD) is an inflammatory disease of unknown etiology. IBD is an autoimmune disease characterized by excessive activation of the adaptive immune response. Various factors including genetic factors alter the intestinal flora and trigger an inflammatory reaction, which activates T cells, B cells, mast cells, macrophages and microglia, smooth muscle cells and fibroblasts in the colon, thereby inducing mucosal disruption. Epithelial and endothelial damage release chemotactic factors promoting recruitment and activation of inflammatory cells, and release of various cytokines.

In some embodiments, compounds described herein are useful for treating (e.g., lessening the severity of such as by delaying onset and/or reducing degree and/or frequency of one or more features of) IBD. In some embodiments, provided compounds are considered effective for treating IBD if one or more of the following outcomes is observed in a patient or population thereof improved body weight, reduced severity and/or frequency of diarrhea, reduced severity and/or frequency of fever, reduced severity and/or frequency of fatigue, reduced severity and/or frequency of abdominal pain, reduced frequency of defecation, reduced severity and/or frequency of abdominal cramping, reduced severity and/or frequency of bloody stool, improved appetite, reduced severity and/or frequency of signs and symptoms of IBD generally, occurrence of major clinical response, reduced intestinal structural damage, improved overall physical function, maintained clinical remission (e.g., over a particular period of time), improved Crohn's disease activity index (CDAI), improved Mayo score (e.g., in a patient with ulcerative colitis), reduced levels of one or more circulating cytokines (e.g., IL-1, TNF-α, or PDGF), and/or occurrence of clinical remission (e.g., in a patient with Crohn's disease or in a patient with ulcerative colitis).

### Provided Compounds & Methods

PCT Application No. PCT/US2013/023324, filed Jan. 26, 2013, and published as WO2013/112959 on Aug. 1, 2013, the entirety of which is hereby incorporated by reference, describes certain antifibrotic compounds. These compounds are tyrosine kinase inhibitors with demonstrated activity against platelet-derived growth factor receptors (PDGFR) and vascular endothelial growth factor receptors (VEGFR2). WO2013/112959 describes certain indications with a fibrotic component for which these compounds are useful as therapy.

The present disclosure encompasses the recognition that the compounds described in WO2013/112959 are surprisingly also useful for the treatment of IBD (e.g., according to methods described herein). As described above, IBD is primarily an inflammatory autoimmune disease, and, as such, it was not known if anti-fibrotic compounds (such as those described in WO2013/112959) would be useful for treating IBD. Yet, as demonstrated below in the Examples, provided compounds display therapeutic benefits in IBD animal models, potentially beyond mere anti-fibrotic effects.

There remains a need for new IBD therapies. Currently, anti-inflammatory drugs, including, 5-aminosalicylic acid (5-ASA)-based preparations, are often the first line of therapy in IBD (Segars, L. W., et al., Clin. Pharm. 1992 June; 11(6):514-28). Anti-TNFα antibodies such as infliximab (REMICADE) and adalimumab (HUMIRA) are also being used. Nevertheless, patients treated with HUMIRA are at increased risk for developing serious infections and lymphoma (Dulai, P. S., et al., Clin. Gastroenterol. Hepatol. 2014 September; 12(9):1443-51). Corticosteroids, other immuno-suppressants and antibiotics exhibit multiple side effects but relatively poor treatment responses (Kopylov, U. and Seidman, E., Adv. Gastroenterol. 2016 July; 9(4):513-26; Waljee, A. K., et al., PLoS One. 2016 Jun. 23; 11(6):e0158017; Cosnes, J., et al., Gut. 54 (2005) pp. 237-241). Despite substantial advances in its management, IBD still displays a chronic inflammatory course, and the incidence of stricture formation and stenosis secondary to inflammation has not significantly changed during the last 25 years. In contrast to progress of anti-inflammatory therapies in IBD, relatively minor progress has occurred with respect to a therapeutic approach to intestinal fibrosis. Anti-inflammatory therapies to date do not completely prevent fibrosis or reverse established fibrosis. Bowel resection and stricture-plasty remain the basic interventions for complications secondary to intestinal fibrosis. Less invasive procedures for treatment of strictures are increasingly used, such as balloon dilatation, polyvinyl over-the-guide wire dilatation and injection of glucocorticoids into the strictures after dilatation. However, the long-term efficacy of these measures is limited by the frequent recurrence of the problem.

In some embodiments, the present disclosure provides methods for treating IBD, comprising administering to a subject in need thereof a compound of Formula (I):

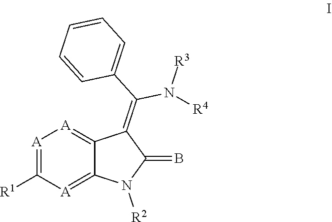

or a pharmaceutically acceptable salt thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂,
NR⁶R⁷, NR⁶COR⁷ or NR⁶SO₂R⁷;
R⁵ is a lower alkyl group;
R⁶ and R⁷ are independently hydrogen, alkyl, cycloalkyl, heterocycle, cycloalkylalkyl, alkylcycloalkylalkyl, heterocycloalkyl or alkylheterocycloalkyl, which may be optionally substituted with alkyl, OR⁸, COOR⁸, NR⁸R⁹, or NHCOR⁸;
R⁸ and R⁹ are independently H or a lower alkyl group;
A is N or CH, wherein one A is nitrogen; and
B is O or S.

In some embodiments of Formula (I), R² is hydrogen.

In some embodiments of Formula (I), R² is a carbamate prodrug moiety.

In some embodiments of Formula (I), R² is an amide prodrug moiety.

In some embodiments of Formula (I), R³ is hydrogen.

In some embodiments of Formula (I), R⁴ is aryl, such as phenyl. In some embodiments, R⁴ is aryl optionally substituted with one or more lower alkyl, halogen, OR⁶, NO₂, CN, NH₂, NR⁶R⁷, NR⁶COR⁷ or NR⁶SO₂R⁷. In some embodiments, R⁴ is phenyl optionally substituted with NR⁶COR⁷. In some embodiments, R⁴ is phenyl substituted with NR⁶COR⁷.

In some embodiments of Formula (I), R⁵ is lower alkyl, such as methyl.

In some embodiments of Formula (I), R⁶ is alkyl, such as methyl.

In some embodiments of Formula (I), R⁷ is alkylheterocycloalkyl, such as methylpiperazinylmethyl. In some embodiments, R⁷ is

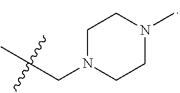 [SMILES: *CN1CCN(C)CC1]

In some embodiments of Formula (I), R⁸ is lower alkyl, such as methyl.

In some embodiments of Formula (I), R⁹ is hydrogen.

In some embodiments of Formula (I), the A at position 4 is N, and the other occurrences of A are C. In some embodiments, the A at position 5 is N, and the other occurrences of A are C. In some embodiments, the A at position 7 is N, and the other occurrences of A are C.

In some embodiments of Formula (I), B is O. In some embodiments, B is S.

Non-limiting examples of compounds of formula (I) include: (Z)-methyl 3-(((4-(N-methyl-2-(4-methylpiperazin-1-yl)acetamido)phenyl)amino)(phenyl)methylene)-2-oxo-2,3-dihydro-1H-pyrrolo[2,3-b]pyridine-6-carboxylate; (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[2,3-b]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[2,3-b]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[2,3-b]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[2,3-b]pyridine-6-carboxylate; (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[2,3-b]pyridine-6-carboxylate; (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-c]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-c]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-c]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[3,2-c]pyridine-6-carboxylate; (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-c]pyridine-6-carboxylate; (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[3,2-c]pyridine-6-carboxylate; (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-b]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-b]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-b]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[3,2-b]pyridine-6-carboxylate; (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-b]pyridine-6-carboxylate; and (Z)-methyl 3-(((4-(methyl(2-(4-methylpiperazin-1-yl)ethyl)amino)phenyl)amino)(phenyl)methylene)-2-oxo-2,3-dihydro-TH-pyrrolo[3,2-b]pyridine-6-carboxylate.

In some embodiments, the present disclosure provides methods of treating IBD, comprising administering to a subject in need thereof a compound of Formula (II):

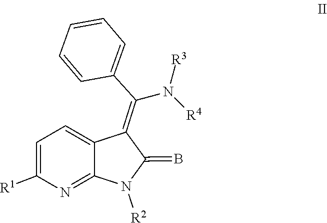

or a pharmaceutically acceptable salt thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷;
R⁵ is a lower alkyl group;
R⁶ and R⁷ are independently hydrogen, alkyl, cycloalkyl, heterocycle, cycloalkylalkyl, alkylcycloalkylalkyl, heterocycloalkyl or alkylheterocycloalkyl, which may be optionally substituted with alkyl, OR⁸, COOR⁸, NR⁸R⁹, or NHCOR⁸;
R⁸ and R⁹ are independently H or a lower alkyl group; and
B is O or S.

In some embodiments of Formula (II), R² is hydrogen.

In some embodiments of Formula (II), R² is a carbamate prodrug moiety.

In some embodiments of Formula (II), R² is an amide prodrug moiety.

In some embodiments of Formula (II), R³ is hydrogen.

In some embodiments of Formula (II), R⁴ is aryl, such as phenyl. In some embodiments, R⁴ is aryl optionally substituted with one or more lower alkyl, halogen, OR⁶, NO₂, CN, NH₂, NR⁶R⁷, NR⁶COR⁷ or NR⁶SO₂R⁷. In some embodiments, R⁴ is phenyl optionally substituted with NR⁶COR⁷. In some embodiments, R⁴ is phenyl substituted with NR⁶COR⁷.

In some embodiments of Formula (II), R⁵ is lower alkyl, such as methyl.

In some embodiments of Formula (II), R⁶ is alkyl, such as methyl.

In some embodiments of Formula (II), R⁷ is alkylheterocycloalkyl, such as methylpiperazinylmethyl. In some embodiments, R⁷ is

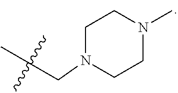 [SMILES: *CCN1CCN(C)CC1]

In some embodiments of Formula (II), R⁸ is lower alkyl, such as methyl.

In some embodiments of Formula (II), R⁹ is hydrogen.

In some embodiments of Formula (II), B is O. In some embodiments, B is S.

Non-limiting examples of compounds of formula (II) include: (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[2,3-b]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[2,3-b]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[2,3-b]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[2,3-b]pyridine-6-carboxylate; (Z)-methyl 3-(((4-(N-methyl-2-(4-methylpiperazin-1-yl)acetamido)phenyl)amino)(phenyl)methylene)-2-oxo-2,3-dihydro-1H-pyrrolo[2,3-b]pyridine-6-carboxylate; and (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[2,3-b]pyridine-6-carboxylate.

In some embodiments, the present disclosure provides methods of treating IBD, comprising administering a compound of Formula (III):

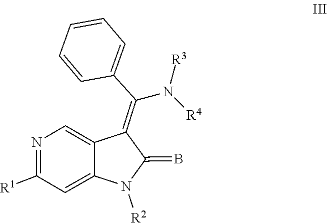

or a pharmaceutically acceptable salt thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷;
R⁵ is a lower alkyl group;
R⁶ and R⁷ are independently hydrogen, alkyl, cycloalkyl, heterocycle, cycloalkylalkyl, alkylcycloalkylalkyl, heterocycloalkyl or alkylheterocycloalkyl, which may be optionally substituted with alkyl, OR⁸, COOR⁸, NR⁸R⁹, or NHCOR⁸;
R⁸ and R⁹ are independently H or a lower alkyl group; and
B is O or S.

In some embodiments of Formula (III), R² is hydrogen.

In some embodiments of Formula (III), R² is a carbamate prodrug moiety.

In some embodiments of Formula (III), R² is an amide prodrug moiety.

In some embodiments of Formula (III), R³ is hydrogen.

In some embodiments of Formula (III), R⁴ is aryl, such as phenyl. In some embodiments, R⁴ is aryl optionally substituted with one or more lower alkyl, halogen, OR⁶, NO₂, CN, NH₂, NR⁶R⁷, NR⁶COR⁷ or NR⁶SO₂R⁷. In some embodiments, R⁴ is phenyl optionally substituted with NR⁶COR⁷. In some embodiments, R⁴ is phenyl substituted with NR⁶COR⁷.

In some embodiments of Formula (III), R⁵ is lower alkyl, such as methyl.

In some embodiments of Formula (III), R⁶ is alkyl, such as methyl.

In some embodiments of Formula (III), R⁷ is alkylheterocycloalkyl, such as methylpiperazinylmethyl. In some embodiments, R⁷ is

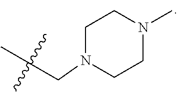 [SMILES: *CCN1CCN(C)CC1]

In some embodiments of Formula (III), R⁸ is lower alkyl, such as methyl.

In some embodiments of Formula (III), R⁹ is hydrogen.

In some embodiments of Formula (III), B is O. In some embodiments, B is S.

Non-limiting examples of compounds of formula (III) include: (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-c]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-c]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-c]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[3,2-c]pyridine-6-carboxylate; (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-c]pyridine-6-carboxylate; and (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[3,2-c]pyridine-6-carboxylate.

In some embodiments, the present disclosure provides methods of treating IBD, comprising administering a compound of Formula (IV):

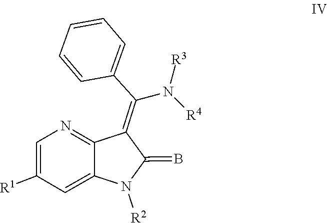

or a pharmaceutically acceptable salt thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷;
R⁵ is lower alkyl group;
R⁶ and R⁷ are independently hydrogen, alkyl, cycloalkyl, heterocycle, cycloalkylalkyl, alkylcycloalkylalkyl, heterocycloalkyl or alkylheterocycloalkyl, which may be optionally substituted with alkyl, OR⁸, COOR⁸, NR⁸R⁹, or NHCOR⁸;
R⁸ and R⁹ are independently H or a lower alkyl group; and
B is O or S.

In some embodiments of Formula (IV), R² is hydrogen.

In some embodiments of Formula (IV), R² is a carbamate prodrug moiety.

In some embodiments of Formula (IV), R² is an amide prodrug moiety.

In some embodiments of Formula (IV), R³ is hydrogen.

In some embodiments of Formula (IV), R⁴ is aryl, such as phenyl. In some embodiments, R⁴ is aryl optionally substituted with one or more lower alkyl, halogen, OR⁶, NO₂, CN, NH₂, NR⁶R⁷, NR⁶COR⁷ or NR⁶SO₂R⁷. In some embodiments, R⁴ is phenyl optionally substituted with NR⁶COR⁷. In some embodiments, R⁴ is phenyl substituted with NR⁶COR⁷.

In some embodiments of Formula (IV), R⁵ is lower alkyl, such as methyl.

In some embodiments of Formula (IV), R⁶ is alkyl, such as methyl.

In some embodiments of Formula (IV), R⁷ is alkylheterocycloalkyl, such as methylpiperazinylmethyl. In some embodiments, R⁷ is

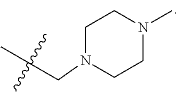 [SMILES: *CCN1CCN(C)CC1]

In some embodiments of Formula (IV), R⁸ is lower alkyl, such as methyl.

In some embodiments of Formula (IV), R⁹ is hydrogen.

In some embodiments of Formula (IV), B is O. In some embodiments, B is S.

Non-limiting examples of compounds of formula (IV) include: (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-b]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-b]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-b]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[3,2-b]pyridine-6-carboxylate; (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-b]pyridine-6-carboxylate; and (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[3,2-b]pyridine-6-carboxylate.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomeric, and geometric (or conformational) mixtures of provided compounds are within the scope of the disclosure. Unless otherwise stated, all tautomeric forms (pyrazoles, pyridones and enols, etc.) of provided compounds are within the scope of the disclosure.

In addition to the above-mentioned compounds per se, this disclosure also encompasses pharmaceutically acceptable derivatives of these compounds, as well as compositions comprising one or more compounds of the disclosure and one or more pharmaceutically acceptable excipients or additives.

In some embodiments, compounds are provided and/or utilized in the form a salt (e.g., a pharmaceutically acceptable salt). As used herein, the term “pharmaceutically acceptable salt” refers to those salts which are, within the scope of sound medical judgment, suitable for use in contact with the tissues of humans and/or lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the disclosure, or separately by reacting a free base or free acid functional group with a suitable reagent, as described generally below. For example, a free base functional group can be reacted with a suitable acid. Furthermore, where the compounds of the disclosure carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of a basic group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, compounds are provided and/or utilized in the form of a pharmaceutically acceptable derivative, including but not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof.

As used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

The term “pharmaceutically acceptable prodrugs” as used herein refers to those prodrugs of the compounds of the present disclosure which are, within the scope of sound medical judgment, suitable for use in contact with the issues of humans and/or lower animals with undue toxicity, irritation, allergic response, and the like, commensurate with a reasonable benefit/risk ratio, and effective for their intended use. The term “prodrug” refers to compounds that are rapidly transformed in vivo to yield the parent compound (e.g., a compound of Formula (I)), for example by hydrolysis in blood, or N-demethylation of a compound. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference. By way of other non-limiting examples, carbamate and amide prodrugs of compounds of Formulae (I)-(IV) are embodied herein, such as those discussed in Rautio et al., 2008, Nature Rev Drug Discov 7:255-70; Jordan et al., 2003, Bioorg Med Chem 10:2625-33 and Hay et al., 2003, J Med Chem 46:5533-45.

Compounds of the disclosure may be prepared by crystallization of a compound of Formula (I), (II), (III) and (IV) under different conditions and may exist as one or a combination of polymorphs of compound of general formulas (I), (II), (III) and (IV). For example, different polymorphs may be identified and/or prepared using different solvents, or different mixtures of solvents for recrystallization; by performing crystallizations at different temperatures; or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other techniques. Thus, the present disclosure encompasses provided compounds, their derivatives, their tautomeric and geometrical isomeric forms, their stereoisomers, their positional isomer, their polymorphs, their pharmaceutically acceptable salts, their pharmaceutically acceptable solvates and pharmaceutically acceptable compositions containing them.

### Pharmaceutical Compositions

As discussed above this disclosure provides compounds that are useful for the treatment of inflammatory bowel disease, such as Crohn's disease and ulcerative colitis. Accordingly, in another aspect of the present disclosure, pharmaceutical compositions are provided for use in treatment of IBD, which comprise any one or more of the compounds described herein (or a prodrug, pharmaceutically acceptable salt or other pharmaceutically acceptable derivative thereof), and optionally comprise a pharmaceutically acceptable carrier. In some embodiments, provided pharmaceutical compositions are solid compositions, such as a tablet or capsule.

As described above, in some embodiments, the pharmaceutical compositions of the present disclosure for use in treating IBD additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with compounds of the disclosure, such as by producing any undesirable biological effect or otherwise interacting in a deleterious manner with any other component(s) of the pharmaceutical composition, its use is contemplated to be within the scope of this disclosure. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil, safflower oil, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; pyrogenfree water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants.

Liquid dosage forms for oral administration include, but are not limited to, pharmaceutically acceptable emulsions, microemulsions, solutions, suspensions, syrups and elixirs. In addition to the active compounds, liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut (peanut), corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions which are compatible with body tissues.

Compositions for rectal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum and release the active compound.

Solid dosage forms for oral administration include capsules, tablets, pills, powders, and granules. In such solid dosage forms, the active compound is optionally mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules, optionally using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents. In some embodiments, solid dosage forms comprise a controlled release coating, such that, e.g., they release the active ingredient(s) only, or preferentially, in a certain part of the intestinal tract, such as the colon, optionally, in a delayed manner. Examples of embedding compositions that can be used include polymeric substances and waxes.

The active compounds can also be in micro-encapsulated form with one or more excipients as noted above. In such solid dosage forms, the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents.

In some embodiments, provided compounds can be co-precipitated with one or more agents such as mannitol, a combination of mannitol and lactobionic acid, a combination of mannitol and gluconic acid, a combination of mannitol and methanesulfonic acid, a combination of microcrystalline cellulose and oleic acid or a combination of pregelatinized starch and oleic acid. The foregoing examples of one or more agents to aid in preparing formulations of provided compounds are merely illustrative and non-limiting.

In some embodiments, the present disclosure encompasses the recognition that delivery of provided compounds to the colon may be desirable for treating IBD. For example, in some embodiments, provided compounds are formulated to control release of the compound, such that a therapeutically effective amount is delivered to the colon. In some embodiments, provided compounds are formulated as an oral controlled-release composition. In some embodiments, provided compounds are formulated as a suppository. Without wishing to be bound by theory, the present disclosure recognizes that compositions which provide a compound disclosed herein with low oral bioavailability may be better suited to deliver said compound to the colon, than compositions that provide the compound with high oral bioavailability.

Various strategies for colon-targeted drug delivery are known in the art. See, e.g., Amidon, S., et al., AAPS PharmSciTech, Vol. 16, No. 4, August 2015, pp 731-741; Philip, A. K. and Philip, B. Oman Med. J. 2010, Vol. 25, Issue 2, pp. 70-78; Kotla, N. G., et al., Int. J. Nanomedicine, 2016:11, 1089-1095, each of which is hereby incorporated by reference.

In some embodiments, the present disclosure provides a solid dosage form comprising a compound described herein or a pharmaceutically acceptable salt thereof. In some embodiments, the solid dosage form is a capsule or tablet.

In some embodiments, a pharmaceutical composition is provided comprising a compound described herein and a carrier, excipient or diluent, wherein the pharmaceutical composition, upon administration to a subject, releases a particular amount (e.g., a therapeutically effective amount) of the compound to the colon. In some embodiments, a provided pharmaceutical composition delivers >95%, >90%, >85%, >80%, >75%, >70%, >65%, >60%, >55%, >50%, >45%, >40%, >35%, >30%, >25%, or >20% of the compound to the colon, relative to the total amount of compound in the pharmaceutical composition. The amount of compound delivered to the colon can be determined by any suitable methods known in the art. For example, absorption of drugs in the colon can be monitored using colonoscopy and intubation and/or with gamma scintigraphy. See, e.g., Philip, A. K. and Philip, B. Oman Med. J. 2010, Vol. 25, Issue 2, pp. 70-78.

Without wishing to be bound by theory, the present disclosure also encompasses the recognition that oral administration of provided compounds (e.g., in such a way to provide high oral bioavailability) may be desirable for treating IBD. In some embodiments, a provided pharmaceutical composition, upon administration to a subject, achieves an oral bioavailability of greater than about 5%, greater than about 10%, greater than about 15%, or greater than about 20%.

In some embodiments, the present disclosure provides a pharmaceutical composition comprising a provided compound combined with an appropriate topical cream base. In some such embodiments, the cream composition comprises from about 5 wt % to about 75 wt % of the provided compound. In some embodiments, the cream composition comprises from about 7.5 wt % to about 50 wt % of the provided compound. In some embodiments, the cream composition comprises from about 10 wt % to about 35 wt % of the provided compound. In some embodiments, the cream composition comprises from about 15 wt % to about 25 wt % of the provided compound. In some embodiments, a cream composition described herein can be applied directly to the anatomical tissue within the colon.

In some embodiments, the present disclosure provides a pharmaceutical composition comprising a provided compound combined with an appropriate topical gel base. In some embodiments, the gel composition comprises from about 5 wt % to about 75 wt % of the provided compound. In some embodiments, the gel composition comprises from about 7.5 wt % to about 50 wt % of the provided compound. In some embodiments, the gel composition comprises from about 10 wt % to about 35 wt % of the provided compound. In some embodiments, the gel composition comprises from about 15 wt % to about 25 wt % of the provided compound. In some embodiments, a gel composition described herein can be applied directly to the anatomical tissue within the colon.

In some embodiments, the present disclosure provides a pharmaceutical composition comprising a provided compound combined with an appropriate topical lotion base. In some embodiments, the lotion composition comprises from about 5 wt % to about 75 wt % of the provided compound. In some embodiments, the lotion composition comprises from about 7.5 wt % to about 50 wt % of the provided compound. In some embodiments, the lotion composition comprises from about 10 wt % to about 35 wt % of the provided compound. In some embodiments, the lotion composition comprises from about 15 wt % to about 25 wt % of the provided composition. In some embodiments, a lotion composition described herein can be applied directly to the anatomical tissue within the colon.

In some embodiments, the present disclosure provides a pharmaceutical composition comprising a provided compound combined with an appropriate topical foam base. In some embodiments, the foam composition comprises from about 5 wt % to about 75 wt % of the provided compound. In some embodiments, the foam composition comprises from about 7.5 wt % to about 50 wt % of the provided compound. In some embodiments, the foam composition comprises from about 10 wt % to about 35 wt % of the provided compound. In some embodiments, the foam composition comprises from about 15 wt % to about 25 wt % of the provided compound. In some embodiments, a foam composition described herein can be applied directly to the anatomical tissue within the colon.

In some embodiments, the present disclosure also provides suppository compositions comprising provided compounds. Suppositories are commonly administered to the rectum as a means for dispensing various active ingredients or medicaments. Suppositories are made in various shapes including oviform, globular, conical and bullet shapes, and in various sizes. Suppositories typically weigh in the range of 1 to 5 grams. Suppositories can be solid bodies composed of a mixture of a suitable suppository base material and the active ingredients or medicaments. Alternatively, suppositories can be made with a solid outer wall of suppository base material enclosing non-solid active ingredients or medicaments. Typically, the suppository base materials are selected so that they dissolve or melt when exposed to moisture (e.g., bodily fluids) and/or heat (e.g., body temperature) of the vagina or rectum (i.e., rectal or anal canal), thereby releasing the active ingredients or medicaments into the body. Suitable suppository base materials include oleaginous (fatty) base materials, such as cocoa butter, theobroma oil and synthetic triglycerides, or water soluble or water miscible base materials, such as glycerinated gelatin and polyethylene glycol (PEG) polymers. It is preferred that the base materials be non-toxic, non-irritating, inert, and biocompatible. Suppositories suitable for use in an aspect of the present disclosure can be prepared in various ways according to conventional methods for preparing suppositories including compression molding and fusion molding.

In some embodiments, the present disclosure provides enema compositions comprising provided compounds, e.g., for rectal administration. Enema compositions can be prepared from a provided compound and a suitable liquid carrier (e.g., aqueous buffer or saline). Enema compositions may further comprise additional ingredients, such as antioxidants and/or preservatives.

The present disclosure also encompasses pharmaceutically acceptable topical formulations of provided compounds, such as topical creams, gels, lotions, and foams described herein. The term “pharmaceutically acceptable topical formulation”, as used herein, means any formulation which is pharmaceutically acceptable for administration of a compound of the disclosure by application of the formulation to the epidermis. In some embodiments of the disclosure, the topical formulation comprises a carrier system. Pharmaceutically effective carriers include, but are not limited to, solvents (e.g., alcohols, poly alcohols, water), creams, lotions, ointments, oils, plasters, liposomes, powders, emulsions, microemulsions, and buffered solutions (e.g., hypotonic or buffered saline) or any other carrier known in the art for topically administering pharmaceuticals. A more complete listing of art-known carriers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In some embodiments, the topical formulations of the disclosure may comprise excipients. Any pharmaceutically acceptable excipient known in the art may be used to prepare the provided pharmaceutically acceptable topical formulations. Examples of excipients that can be included in the topical formulations of the disclosure include, but are not limited to, preservatives, antioxidants, moisturizers, emollients, buffering agents, solubilizing agents, other penetration agents, skin protectants, surfactants, and propellants, and/or additional therapeutic agents used in combination with the active agent. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerine, sorbitol, polyethylene glycols, urea, and propylene glycol. Suitable buffering agents for use with the present disclosure include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the present disclosure include, but are not limited to, vitamin E oil, allatoin, dimethicone, glycerin, petrolatum, and zinc oxide.

In some embodiments, provided compositions may be in the form of ointments, pastes, creams, lotions, gels, powders, solutions, sprays, inhalants or patches. In some embodiments, provided formulations are creams, which may further contain saturated or unsaturated fatty acids such as stearic acid, palmitic acid, oleic acid, palmito-oleic acid, cetyl or oleyl alcohols. In some embodiments, provided cream formulations further comprise stearic acid. Creams of the disclosure may also contain a non-ionic surfactant, for example, polyoxy-40-stearate. In some embodiments, the active component is admixed under sterile conditions with a pharmaceutically acceptable carrier and any needed preservatives or buffers as may be required to provide compositions described herein.

It will also be appreciated that provided compounds and pharmaceutical compositions can be formulated and/or employed in combination therapies, that is, the compounds and pharmaceutical compositions can be formulated with and/or administered concurrently with, prior to, or subsequent to, one or more other desired therapeutics or medical procedures. The particular combination of therapies (e.g., therapeutics and/or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a provided compound may be administered concurrently with another anti-inflammatory agent), or they may achieve different effects (e.g., control of any adverse effects). In some embodiments, provided compositions optionally further comprise one or more additional therapeutic agents. Alternatively, a compound of this disclosure may be administered to a patient in need thereof in combination with the administration of one or more other therapeutic agents. For example, additional therapeutic agents for conjoint administration or inclusion in a pharmaceutical composition with a compound of this disclosure may be an approved agent to treat the same or related indication, or it may be any one of a number of agents undergoing approval in the Food and Drug Administration that ultimately obtain approval for the treatment of any disorder related to IBD.

In some embodiments, co-formulation of provided compounds with any one or more agents useful in the treatment of IBD is embraced herein. In non-limiting examples, one or more compounds of the disclosure may be formulated with at least one cytokine inhibitor, growth factor or other biological, such as an interferon, e.g., alpha interferon, or with another small molecule compound. Non-limiting examples of pharmaceutical agents that may be combined therapeutically with compounds of the disclosure include: antivirals and antifibrotics such as interferon alpha, combination of interferon alpha and ribavirin, Lamivudine, Adefovir dipivoxil and interferon gamma; anticoagulants such as heparin and warfarin; antiplatelets e.g., aspirin, ticlopidine and clopidogrel; other growth factors involved in regeneration, e.g., VEGF and FGF and mimetics of these growth factors; antiapoptotic agents; and motility and morphogenic agents. In some embodiments, the pharmaceutical compositions of the present disclosure further comprise one or more additional therapeutically active ingredients (e.g., anti-inflammatory and/or palliative). For purposes of the invention, the term “palliative” refers to treatment that is focused on the relief of symptoms of a disease and/or side effects of a therapeutic regimen but is not curative. For example, palliative treatment encompasses painkillers, antinausea medications and anti-sickness drugs.

### Administration

As described above, the present disclosure provides methods for the treatment of IBD comprising administering a therapeutically effective amount of a compound of Formula (I), (II), (III) or (IV) as described herein, to a subject in need thereof. It will be appreciated that the compounds and compositions, according to the method of the present disclosure, may be administered using any amount and any route of administration effective for the treatment of IBD. In some embodiments, a therapeutically effective amount of a provided compound is an amount sufficient to modulate fibrosis and/or to modulate inflammation and/or to exhibit a therapeutic effect. The exact amount required will vary from subject to subject, depending on the species, age, and general condition of the subject, the severity of the infection, the particular therapeutic agent, its mode and/or route of administration, and the like.

The compounds of the present disclosure are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present disclosure will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, in some embodiments, after formulation with an appropriate pharmaceutically acceptable carrier in a desired dosage, the pharmaceutical compositions described herein can be administered to humans and other animals orally, rectally, parenterally, intraperitoneally, subcutaneously, or any other effective route of administration. In some embodiments, the compounds of the disclosure may be administered at dosage levels of about 0.001 mg/kg to about 50 mg/kg, preferably from about 0.1 mg/kg to about 10 mg/kg for parenteral administration, or preferably from about 1 mg/kg to about 50 mg/kg, more preferably from about 10 mg/kg to about 50 mg/kg for oral administration, based on a subject's body weight, per day or one or more times a day, to obtain the desired therapeutic effect. It will also be appreciated that dosages smaller than 0.001 mg/kg or greater than 50 mg/kg (for example 50-100 mg/kg) can also be administered to a subject. In some embodiments, compounds are administered orally or parenterally. As noted herein, in some embodiments, delivery to the colon is desired, wherein the compound is substantially delivered to the colon and is minimally orally absorbed. Rectal administration by suppository, foam, cream, gel or enema is also embraced by the disclosure.

### Treatment Kit

In some embodiments, the present disclosure provides a kit for conveniently and effectively carrying out the methods in accordance with the present disclosure. In general, a pharmaceutical pack or kit comprises one or more containers filled with one or more of the ingredients of the pharmaceutical compositions of the disclosure. In some embodiments, such kits are well suited for the delivery of solid oral forms, such as tablets or capsules. In some embodiments, a kit comprises a plurality of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice (e.g., a label) in the form prescribed by a governmental agency regulating the manufacture, use or sale of pharmaceutical products, which notice reflects approval by the agency of manufacture, use or sale for human administration.

### Exemplary Embodiments

The following number embodiments, while non-limiting, are exemplary of certain aspects of the present disclosure.

1. A method for treating inflammatory bowel disease comprising administering to a subject in need an effective amount of a compound as described in Formula (I) below, or a pharmaceutical composition thereof:

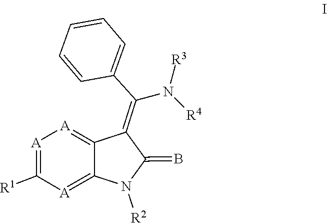

or a pharmaceutically acceptable salt thereof or a prodrug thereof;
where in R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷ moieties;
R⁵ is a lower alkyl group;
R⁶ and R⁷ are independently hydrogen or alkyl, cycloalkyl, heterocycle, cycloalkylalkyl, alkylcycloalkylalkyl, heterocycloalkyl or alkylheterocycloalkyl, which may optionally substituted with alkyl, OR⁸, COOR⁸, NR⁸R⁹, or NCOR⁸ moieties;
R⁸ and R⁹ are independently H or a lower alkyl group;
A is N or CH, wherein one A is nitrogen; and
B is O or S.
   2. The method of embodiment 1 wherein R² is hydrogen.
   3. The method of embodiment 1 wherein R² is a carbamate prodrug moiety or an amide prodrug moiety.
   4. The method of embodiment 1 wherein R³ is hydrogen.
   5. The method of embodiment 1 wherein R⁴ is phenyl.
   6. The method of embodiment 1 wherein R⁵ is methyl.
   7. The method of embodiment 1 wherein R⁶ is methyl.
   8. The method of embodiment 1 wherein R⁷ is methylpiperazinylmethyl.
   9. The method of embodiment 1 having the structure in Formula (II) below:

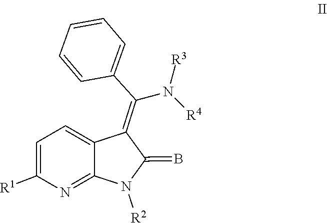

or a pharmaceutically acceptable salt thereof or a prodrug thereof;
where in R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷ moieties;
R⁵ is a lower alkyl group;
R⁶ and R⁷ are independently hydrogen or alkyl, cycloalkyl, heterocycle, cycloalkylalkyl, alkylcycloalkylalkyl, heterocycloalkyl or alkylheterocycloalkyl, which may optionally substituted with alkyl, OR⁸, COOR⁸, NR⁸R⁹, or NCOR⁸ moieties;
R⁸ and R⁹ are independently H or a lower alkyl group; and
B is O or S.
   10. The method of embodiment 9 selected from (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[2,3-b]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[2,3-b]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[2,3-b]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[2,3-b]pyridine-6-carboxylate; (Z)-methyl 3-(((4-(N-methyl-2-(4-methylpiperazin-1-yl)acetamido)phenyl)amino)(phenyl)methylene)-2-oxo-2,3-dihydro-1H-pyrrolo[2,3-b]pyridine-6-carboxylate; and (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[2,3-b]pyridine-6-carboxylate.
   11. The method of embodiment 1 having the structure in Formula (III) below:

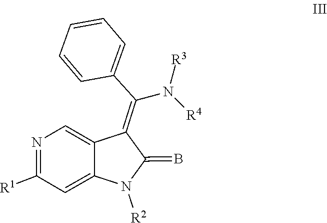

or a pharmaceutically acceptable salt thereof or a prodrug thereof;
where in R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷ moieties;
R⁵ is a lower alkyl group;
R⁶ and R⁷ are independently hydrogen or alkyl, cycloalkyl, heterocycle, cycloalkylalkyl, alkylcycloalkylalkyl, heterocycloalkyl or alkylheterocycloalkyl, which may optionally substituted with alkyl, OR⁸, COOR⁸, NR⁸R⁹, or NCOR⁸ moieties;
R⁸ and R⁹ are independently H or a lower alkyl group; and
B is O or S.
   12. The method of embodiment 11 selected from among (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-c]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-c]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-c]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[3,2-c]pyridine-6-carboxylate; (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-c]pyridine-6-carboxylate; and (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[3,2-c]pyridine-6-carboxylate.
   13. The method of embodiment 1 having the structure in Formula (IV) below:

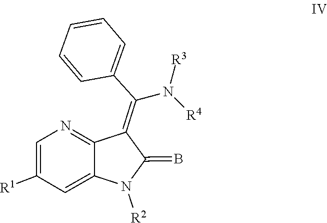

or a pharmaceutically acceptable salt thereof or a prodrug thereof;
where in R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷ moieties;
R⁵ is lower alkyl group;
R⁶ and R⁷ are independently hydrogen or alkyl, cycloalkyl, heterocycle, cycloalkylalkyl, alkylcycloalkylalkyl, heterocycloalkyl or alkylheterocycloalkyl, which may optionally substituted with alkyl, OR⁸, COOR⁸, NR⁸R⁹, or NCOR⁸ moieties;
R⁸ and R⁹ are independently H or a lower alkyl group; and
B is O or S.
   14. The method of embodiment 13 selected from among (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-b]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-b]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-b]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[3,2-b]pyridine-6-carboxylate; (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-b]pyridine-6-carboxylate; and (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[3,2-b]pyridine-6-carboxylate.
   15. The method of any one of embodiments 1-15 wherein the inflammatory bowel disease is ulcerative colitis or Crohn's disease.
   16. The method of embodiment 1 wherein the treating improves body weight in the subject.
   17. The method of embodiment 1 wherein the treating improves diarrhea in the subject.
   18. The method of embodiment 1 wherein the treating reduces fever in the subject.
   19. The method of embodiment 1 wherein the treating reduces fatigue in the subject.
   20. The method of embodiment 1 wherein the treating reduces abdominal pain in the subject.
   21. The method of embodiment 1 wherein the treating reduces defecation in the subject.
   22. The method of embodiment 1 wherein the treating reduces cramping in the subject.
   23. The method of embodiment 1 wherein the treating reduces blood stool in the subject.
   24. The method of embodiment 1 wherein the treating improves appetite in the subject.
   25. The method of embodiment 1 wherein the treating reduces structural damage in the colon.
   26. The method of embodiment 15 wherein the treating improves the Crohn's diseases activity index in a patient with Crohn's disease.
   27. The method of embodiment 15 wherein the treating improves the Mayo score in a subject with ulcerative colitis.
   28. The method of embodiment 1 comprising administering an effective amount of the compound of Formula (I) directly to the colon.
   29. The method of embodiment 28, wherein the compound is administered orally and substantially reaches the colon.
   30. The method of embodiment 28, wherein the compound is administered as a cream directly into or onto the colon.
   31. The method of embodiment 28, wherein the compound is administered as a gel directly onto or into the colon.
   32. The method of embodiment 28, wherein the compound is administered as a lotion directly onto or into the colon.
   33. The method of embodiment 28, wherein the compound is administered as a foam directly onto or into the colon.
   34. The method of embodiment 28, wherein the compound is delivered via suppository to the colon.
   35. The method of embodiment 34, wherein the suppository contains the compound of Formula (I) and the suppository dissolves or melts when exposed to moisture or heat.
   36. The method of embodiment 34, wherein the suppository is composed of a biodegradable material.

### Equivalents

The representative examples that follow are intended to help illustrate the disclosure, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### EXEMPLIFICATION

### Example 1

### Synthesis of Provided Compounds

The synthesis and purification of compounds described herein are disclosed in PCT/US2013/23324, filed Jan. 26, 2013, and published as WO2013/112959 on Aug. 1, 2013, and is incorporated herein by reference in its entirety.

A synthesis of an exemplary compound, (Z)-methyl 3-(((4-(N-methyl-2-(4-methylpiperazin-1-yl)acetamido)phenyl)amino)(phenyl)methylene)-2-oxo-2,3-dihydro-1H-pyrrolo[2,3-b]pyridine-6-carboxylate (“Compound 1”) is provided below.

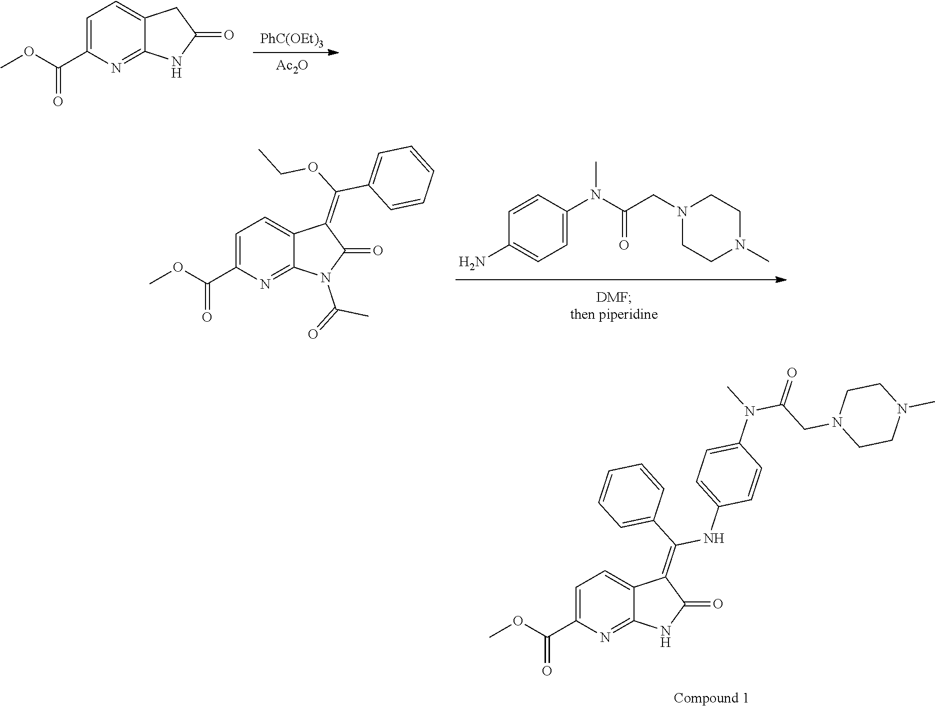

Step 1: To a solution of methyl 2-oxo-2,3-dihydro-1H-pyrrolo[2,3-b]pyridine-6-carboxylate (1 g, 5.20 mmol) in Ac₂O (10 mL) was added triethyl orthobenzoate (3.40 g, 15.59 mmol) at room temperature and the mixture was heated to reflux for 3 h. The reaction mixture was evaporated and the resultant residue was purified by silica gel column chromatography using 5% CH₃OH in dichloromethane as eluent to afford (E)-methyl 1-acetyl-3-(ethoxy(phenyl)methylene)-2-oxo-2,3-dihydro-1H-pyrrolo[2,3-b]pyridine-6-carboxylate as an orange solid. ¹H NMR (CDCl₃, 500 MHz): δ 8.25 (d, J=12.1 Hz, 1H), 8.04 (d, J=12.1 Hz, 1H), 7.53-7.60 (m, 3H), 7.38-7.45 (m, 2H), 4.40 (q, J=7.1 Hz, 2H), 3.99 (s, 3H), 2.63 (s, 3H), 1.42 (t, J=7.1 Hz, 3H).

Step 2: To a solution of (E)-methyl 1-acetyl-3-(ethoxy(phenyl)methylene)-2-oxo-2,3-dihydro-1H-pyrrolo[2,3-b]pyridine-6-carboxylate (2.6 g, 7.10 mmol) in DMF (5 mL) was added N-(4-aminophenyl)-N-methyl-2-(4-methylpiperazin-1-yl)acetamide (1.94 g, 7.43 mmol) at room temperature, and the reaction mixture was heated to 110° C. and stirred for 1 h. The reaction mixture was allowed to cool to room temperature, treated with piperidine (3 mL) and stirred for 30 min. The reaction mixture was evaporated and the resultant residue was purified by silica gel column chromatography using 5% CH₃OH in dichloromethane as eluent to afford (Z)-methyl 3-(((4-(N-methyl-2-(4-methylpiperazin-1-yl)acetamido)phenyl)amino)(phenyl)methylene)-2-oxo-2,3-dihydro-1H-pyrrolo[2,3-b]pyridine-6-carboxylate as yellow solid. MS (ES+): m/z 541.1 (MH⁺).

Other compounds described herein can be prepared using the same procedure, along with general knowledge, including but not limited to:
(Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-c]pyridine-6-carboxylate,
(Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-b]pyridine-6-carboxylate,
(Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[2,3-b]pyridine-6-carboxylate;
(Z)-methyl 3-(((4-((3-(dimethylamino)-3-oxopropyl)(methyl)amino)phenyl)amino)(phenyl)methylene)-2-oxo-2,3-dihydro-1H-pyrrolo[2,3-b]pyridine-6-carboxylate;
(Z)-methyl 3-(((4-(2-(1,1-dioxidothiomorpholino)-N-methylacetamido)phenyl)amino)(phenyl)methylene)-2-oxo-2,3-dihydro-1H-pyrrolo[2,3-b]pyridine-6-carboxylate;
(Z)-methyl 3-(((4-(2-(dimethylamino)-N-methylacetamido)phenyl)amino)(phenyl)methylene)-2-oxo-2,3-dihydro-1H-pyrrolo[2,3-b]pyridine-6-carboxylate;
(Z)-methyl 3-(((4-(methyl(2-(4-methylpiperazin-1-yl)ethyl)amino)phenyl)amino)(phenyl)methylene)-2-oxo-2,3-dihydro-1H-pyrrolo[2,3-b]pyridine-6-carboxylate;
(Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-c]pyridine-6-carboxylate;
(Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-c]pyridine-6-carboxylate;
(Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-c]pyridine-6-carboxylate;
(Z)-methyl 3-(((4-(2-(dimethylamino)-N-methylacetamido)phenyl)amino)(phenyl)methylene)-2-oxo-2,3-dihydro-1H-pyrrolo[3,2-c]pyridine-6-carboxylate;
(Z)-methyl 3-(((4-(methyl(2-(4-methylpiperazin-1-yl)ethyl)amino)phenyl)amino)(phenyl)methylene)-2-oxo-2,3-dihydro-1H-pyrrolo[3,2-c]pyridine-6-carboxylate;
(Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-b]pyridine-6-carboxylate;
(Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-b]pyridine-6-carboxylate;
(Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-b]pyridine-6-carboxylate;
(Z)-methyl 3-(((4-(2-(dimethylamino)-N-methylacetamido)phenyl)amino)(phenyl)methylene)-2-oxo-2,3-dihydro-1H-pyrrolo[3,2-b]pyridine-6-carboxylate; and
(Z)-methyl 3-(((4-(methyl(2-(4-methylpiperazin-1-yl)ethyl)amino)phenyl)amino)(phenyl)methylene)-2-oxo-2,3-dihydro-1H-pyrrolo[3,2-b]pyridine-6-carboxylate.

### Example 2

### Inflammatory Bowel Disease Model: Acetic Acid (AA)

Acetic acid-induced colitis is a common experimental animal model for IBD. Animals treated with acetic acid develop many pathological and histopathological characteristics in common with human ulcerative colitis. Rectal administration of acetic acid damages the mucosal epithelium and induces ulcerative colitis. The severity of the mucosal lesions that develop in acetic acid-induced colitis depends on the acetic acid concentration and the length of exposure. For example, injection of 25% acetic acid (AA) into the gastric lumen causes larger ulcerative lesions than injection of 5% acetic acid. Different concentrations of acetic acid and different exposure times have been reported to induce IBD in a rat model. Transmural necrosis in all layers of the bowel wall, severe neutrophil infiltration of the intestinal tissue, goblet cell depletion, edema, and submucosal ulceration are common manifestations of this model. Bloody diarrhea, reduction of the intestinal mucus, decreased nucleic acid (DNA and RNA) and total protein content, and increased colon weight and vascular permeability have also been observed. MPO activity and MDA levels are elevated, and contents of GSH, superoxide dismutase (SOD) and CAT are significantly reduced in acetic acid-induced colitis. Serum nitrate and lactate dehydrogenase, caspase-3, proinflammatory mediators iNOS, COX-2, IL-1, IL-6, and TNF-α were all significantly increased in acetic acid-treated animals. Acetic acid-induced colitis is also associated with changes NF-κB, inhibitor of κB (IκB) and IκB kinase expression.

All studies were conducted following an approved Institutional Animal Care and Use Committee (IACUC) protocol. All study procedures were conducted by qualified personnel and were in accordance with the approved IACUC protocol and Standard Operation Procedures. All the data reported in the Final Study Report was reviewed by the Investigators or Study Director.

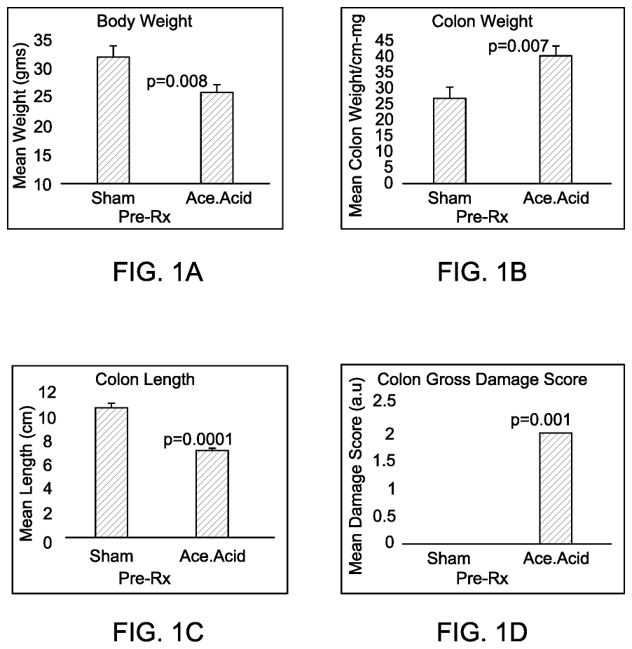

Male CD-1 mice were infused with 4% AA in saline in 150 μL volume through rectal enema under isoflurane anesthesia. A sham group (n=6) received an equivalent volume of saline. After 72 hours of AA (or sham) infusion, a few mice were sacrificed to demonstrate the significant AA-induced IBD pathology including decreased body weight (FIG. 1A), increased colon weight (FIG. 1B), reduced colon length (FIG. 1C), and increased gross morphological colon damage (FIG. 1D).

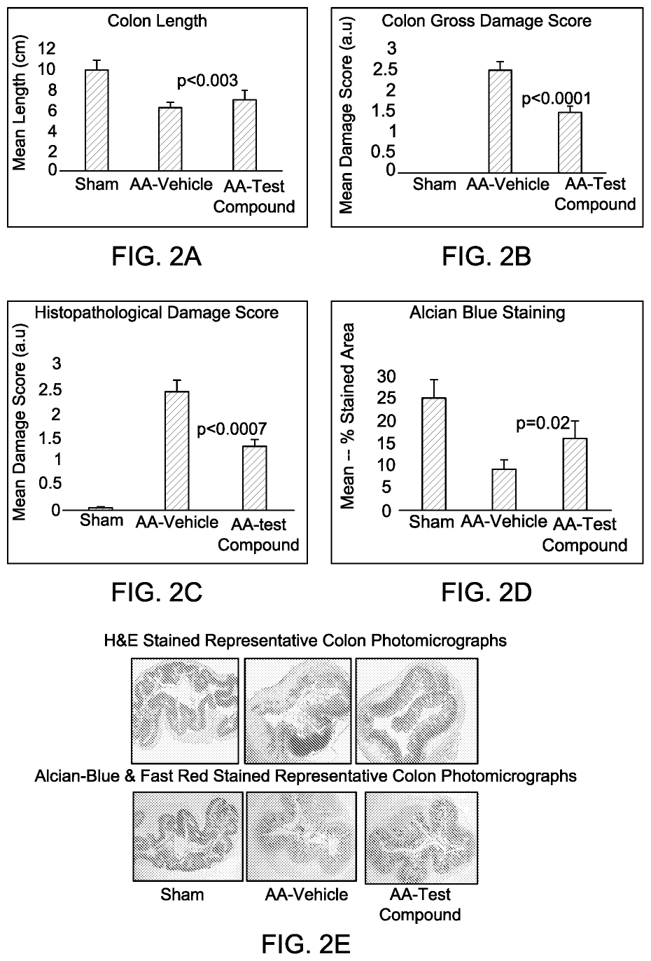

The remaining AA-treated mice (n=9 for each group) were then randomized to vehicle and/or Compound 1 treatment at 50 mg/kg, PO, BID for 3 days. Compound 1 treatment significantly mitigated the decrease in colon length (FIG. 2A), gross morphological colon damage (FIG. 2B) and histopathological colon damage by decreasing mucosal wall thickness and preserving and crypt/villi architecture (FIG. 2C, FIG. 2D, and FIG. 2E) compared to vehicle cohort.

### Example 3

### Inflammatory Bowel Disease Model: 2,4,6-Trinitrobenzenesulfonic Acid (TNBS)

Intracolonic administration of TNBS (2,4,6-trinitrobenzenesulfonic acid)/ethanol induces a severe illness characterized by bloody diarrhea and a dramatic loss of body weight during the first week with increased colon damage score (Mateus, V., et al. Clin. Exp. Gastroenterol. 2018; 11:325-334). TNBS-induced colitis, characteristic of acute colitis and the early phase of IBD, is a commonly utilized animal model in rodents that shares significant properties with human Crohn's disease. Advantages of this model include reproducibility and technical simplicity (Filipescu, I. E., et al. PLoS One 2018 Aug. 23; 13(8):e0202929). The use of TNBS-induced colitis has been valuable in elucidating the mechanisms that mediate disease immune-pathogenesis. TNBS-induced colitis is a well-validated animal model to assess compounds with potential therapeutic effects such as anti-TNFα, corticosteroids, natural compounds and traditional medicine (Mateus 2018; Filipescu 2018; Wirtz, S. et al. Nat. Protoc. 2017 July; 12(7):1295-1309). Compound 1 was therefore tested in the well-established model of TNBS-induced IBD/Acute Colitis.

In this IBD model, 2,4,6-trinitrobenzenesulfonic acid (TNBS) was used to induce disease. The inflammatory bowel disease (IBD)/acute colitis model was conducted in male CD-1 mice, body weights 30-35 grams, from Charles River Laboratories (CRL). After receipt from the vendor, animals were acclimatized to the animal facility at least 5 days following the animal facility standard operating procedures. All animals were fed the normal chow diet, had access to water ad libitum and housed in a single room of the animal facility with a 12:12 hr light-dark cycle and independent ventilation, temperature and humidity control.

Compound 1 was dissolved in distilled water at 0.9 mg/mL, 2.7 mg/mL and 8.1 mg/mL, and 200 μL was administered via oral gavage into each mouse.

Other reagents used include: 2,4,6-Trinitrobenzenesulfonic acid (TNBS) (Sigma, catalogue #P2297), 10% neutral buffered formalin, F4/80 antibody (Cl:A3-1 (MCA497) Bio-Rad), MPO light chain antibody (C-3): sc-390109, anti-rat secondary antibody sigma-Catalogue #AP136P), anti-mouse immunoglobulins (Sigma, stable diaminobenzidene (DAB-catalog number: 750118, hydrogen peroxide (H₂O₂), BSA, PBS and Triton X-100.

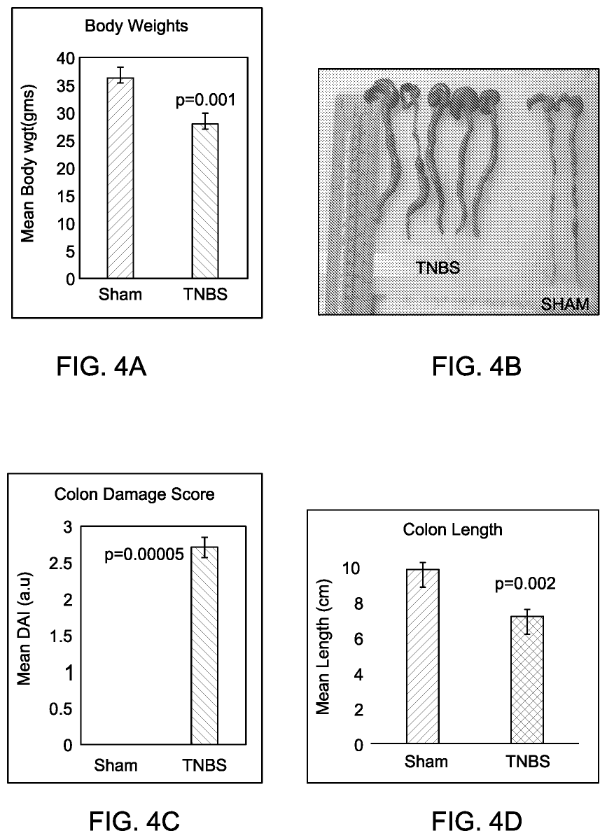

Male CD-1 mice were starved overnight. Then, mice (n=33) were infused with 5% TNBS (2 mg/mouse) in 50% ethanol in a 100 μL volume via rectal enema under isoflurane anesthesia. A sham group (n=6) received an equal volume of 50% ethanol. At 72 hours (3 days) following TNBS (or sham) infusion, a few mice (n=5) were sacrificed to demonstrate the significant TNBS-induced colonic inflammation and pathology, including decreased body weight (FIG. 4A), reduced colon length (FIG. 4B and FIG. 4D) and increased colon gross morphological macroscopic damage score (FIG. 4C) compared to sham (n=2). Remaining Sham (n=4) and TNBS mice (n=7/group) were randomized to vehicle or Compound 1 treatment at 5 mg/kg, 15 mg/kg, and 45 mg/kg, PO, BID for 4 days.

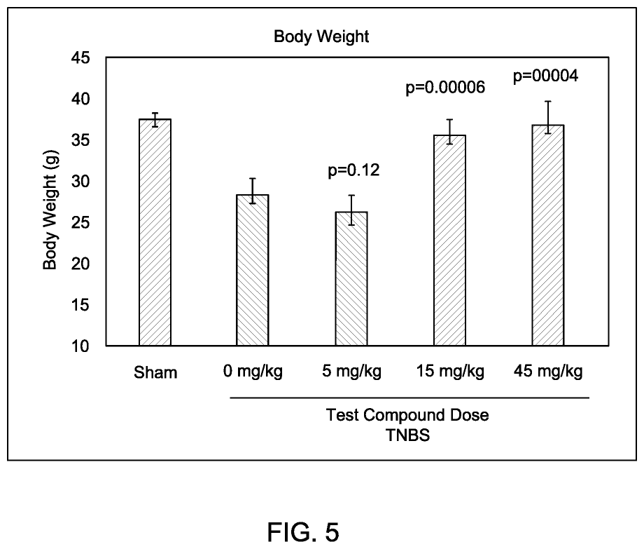

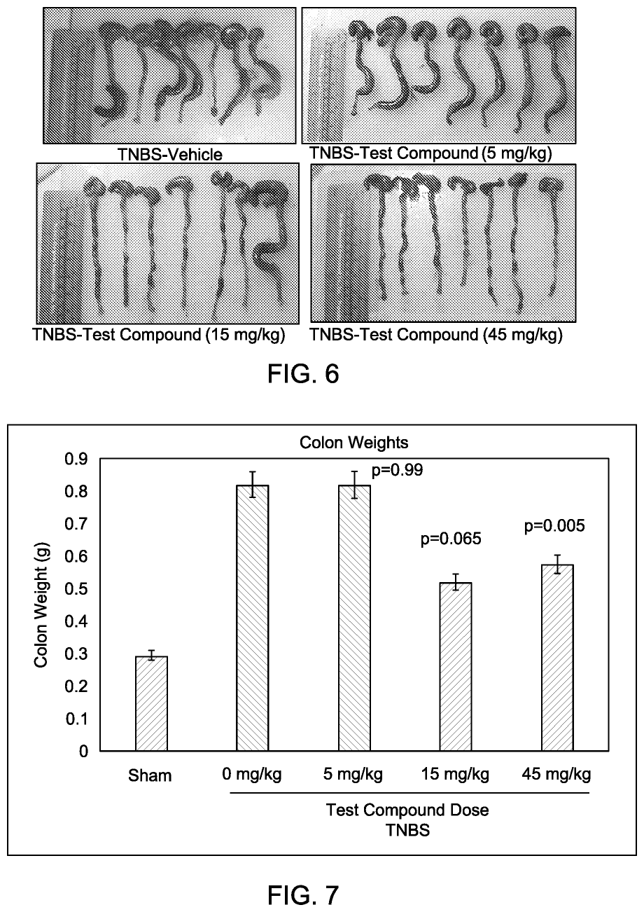

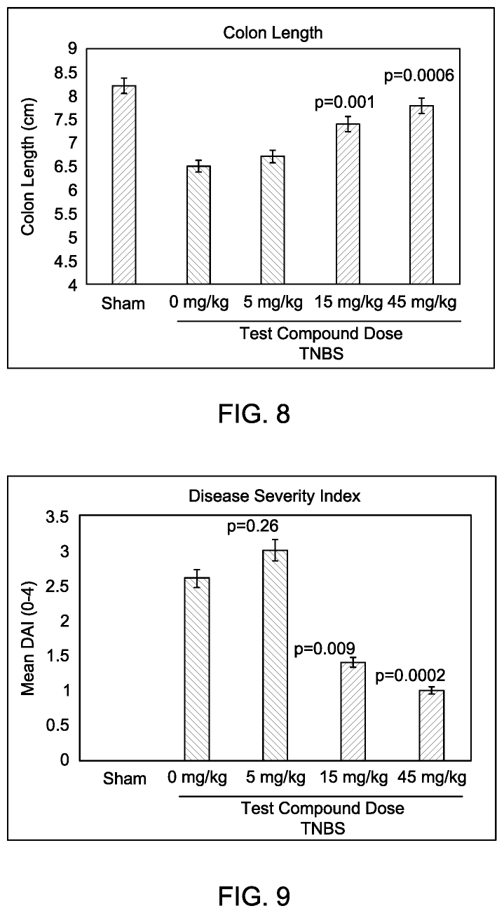

After 4 days of Compound 1 or vehicle treatment, mice were sacrificed. Body weight (FIG. 5), colon length (FIG. 6 and FIG. 8), colon weight (FIG. 7), and gross morphological score/disease severity scores (FIG. 9; normal=0 and severe=4) were recorded. Blood/serum was collected. The mid and distal part of the colon was fixed in 10% neutral buffered formalin and the proximal part of the colon was snap frozen in liquid nitrogen and stored at minus 80° C. until use.

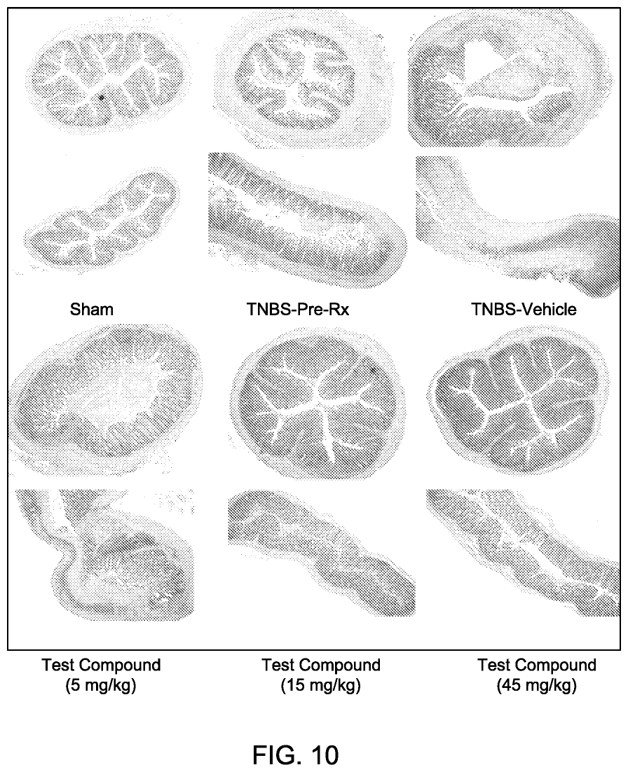

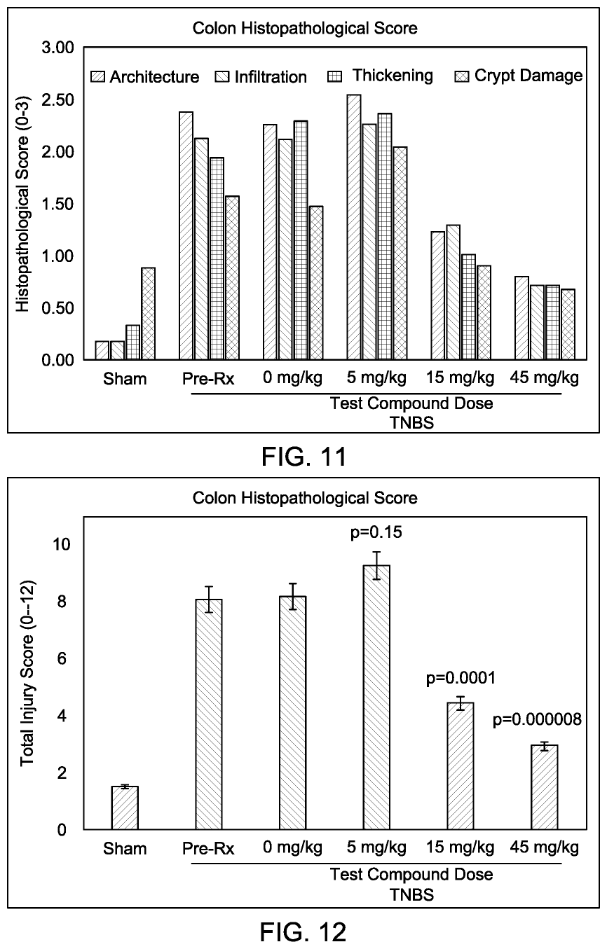

Histological scoring. After overnight fixation in formalin, tissues were dehydrated (graded alcohols) and cleared (xylene) before being embedded in paraffin wax. Sections of tissue were cut and stained with hematoxylin and eosin (H & E) and scored in a blinded manner (FIG. 10). Histological scoring was based on a semi-quantitative scoring system as published (Lab Invest. 1993 August; 69(2):238-49). The following were ranked: the extent of the destruction of normal mucosal architecture (0, normal; 1, 2, and 3, mild, moderate, and extensive damage, respectively), presence and degree of cellular infiltration (0, normal; 1, 2, and 3, mild, moderate, and transmural infiltration, respectively), extent of muscle thickening (0, normal; 1, 2, and 3, mild, moderate, and extensive thickening, respectively), crypt damage/goblet cell depletion (0, no loss, 1, 2, 3 for mild, moderate and extensive loss of goblet cells respectively (FIG. 11). The scores for each feature were summed with a maximum possible colon injury score of 12 (FIG. 12).

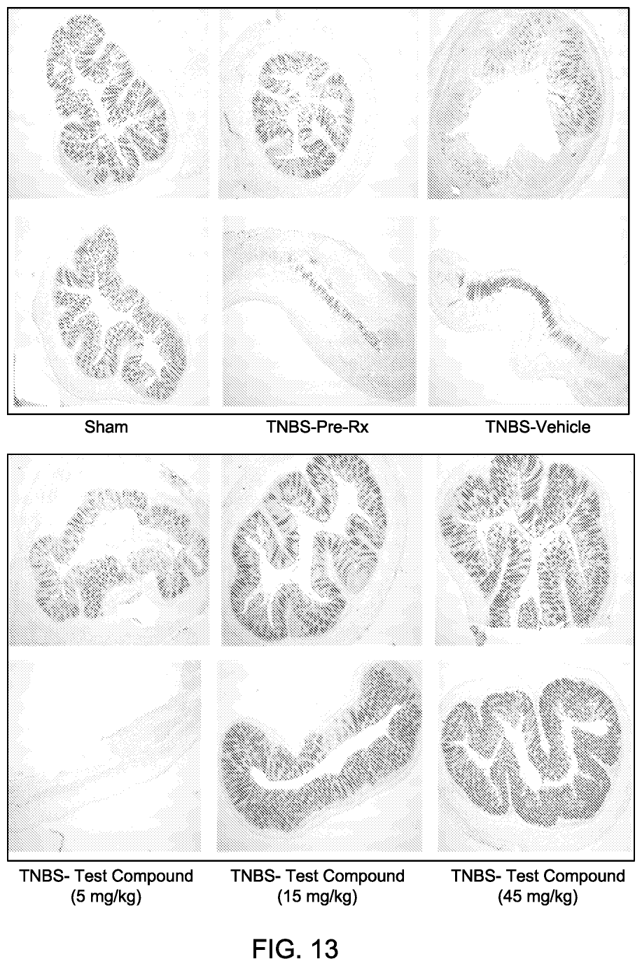

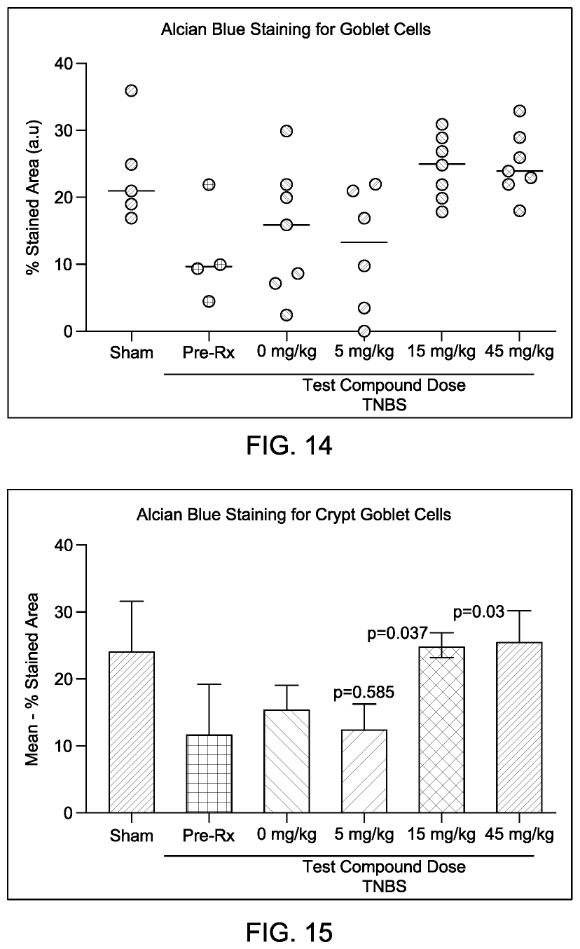

Alcian Blue/Fast red staining. Formalin fixed, paraffin embedded colon tissue sections were used for Alcian blue staining to evaluate crypt damage/goblet cell loss. For Alcian Blue staining, sections were incubated in 1% Alcian Blue in 3% acetic acid, pH 2.5, for 30 minutes and 0.1% nuclear Fast Red for 10 seconds. After staining, sections were dehydrated in ethanol, cleared with xylene and mounted with Cytoseal™ 60. Images were captured on a Nikon microscope. Alcian Blue-positive cells with goblet morphology are well-oriented colonic cross-sections (FIG. 13). The number and maturity of goblet cells, as well as the content of mucus, the intensity of cell infiltrations, and their character, were determined by histological assessment. Bioquant image analysis was performed to quantify blue stained goblet cells for each section and each group to evaluate statistical significance (FIG. 14 and FIG. 15).

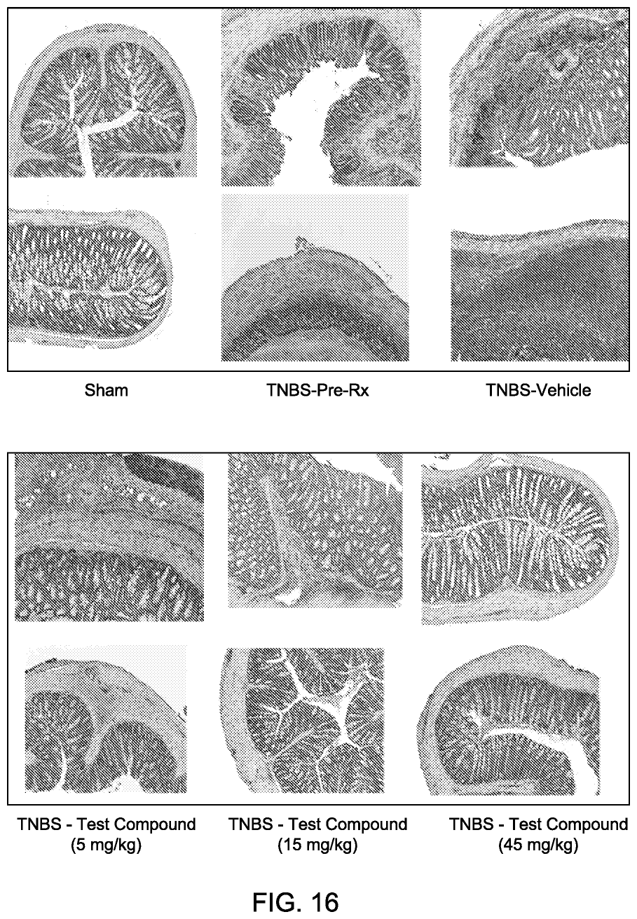

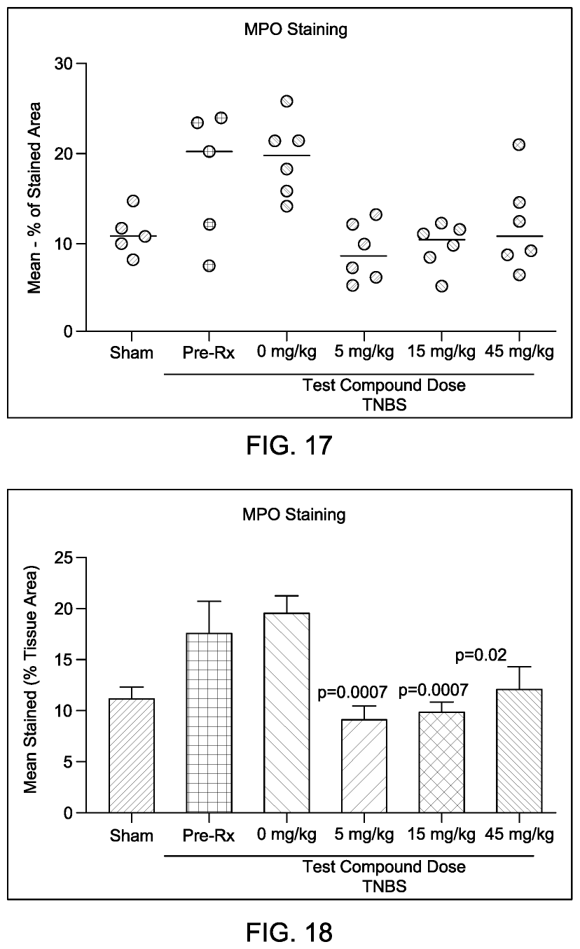

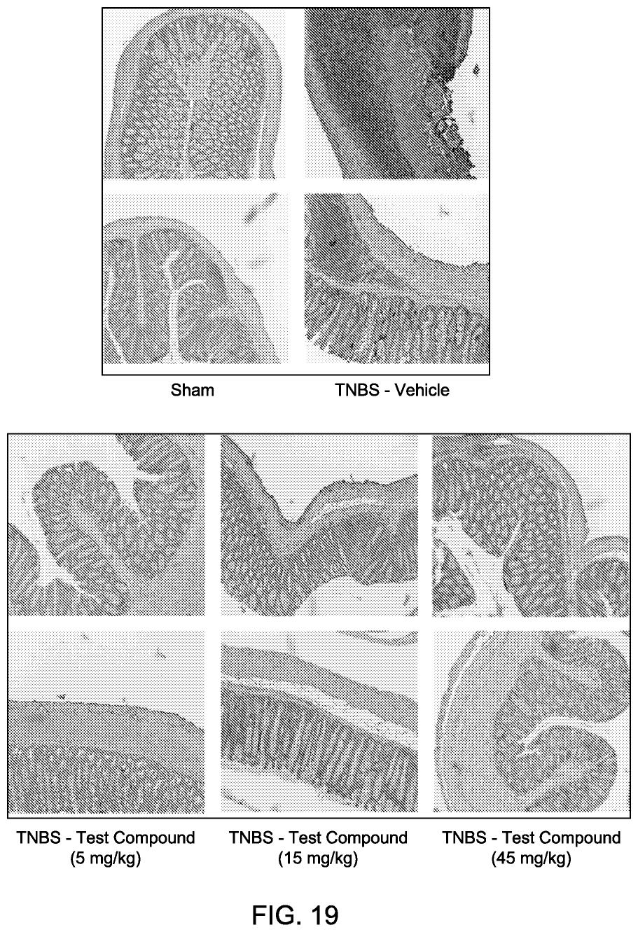

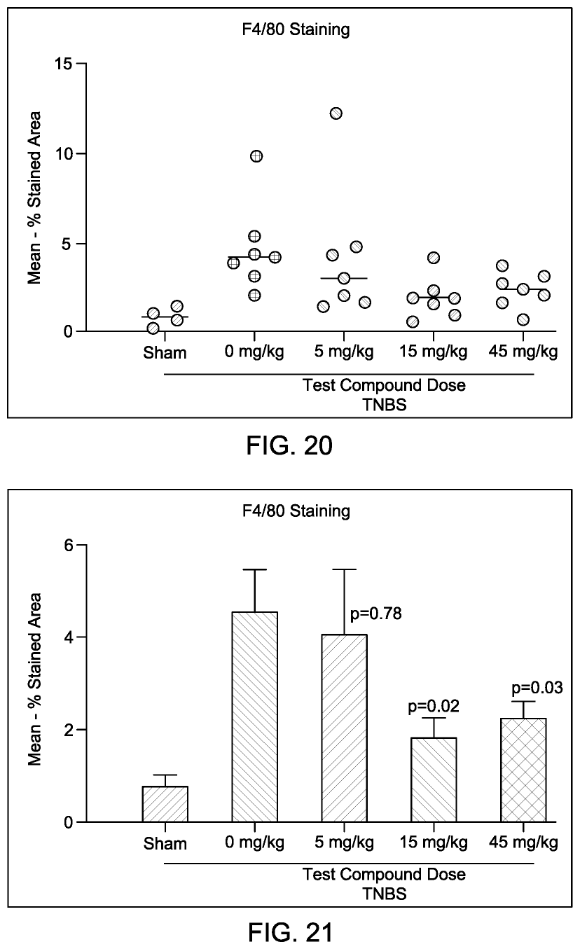

Immunohistochemical (IHC) staining for MPO and F4/80. Colonic tissues were fixed in 4.0% buffered paraformaldehyde, embedded in paraffin and sectioned into 5-μm-thick slices. Endogenous peroxidase was quenched with 3.0% hydrogen peroxide in methanol for 30 min. Sections were further blocked with 3.0% bovine serum albumin (BSA) in PBS, exposed to 0.5% Triton X-100 for 1 h for reducing non-specific antibody binding and incubated with rat anti-mouse mouse F4/80 antibody (Cl:A3-1 (MCA497) Bio-Rad) and mouse MPO light chain antibody (C-3): sc-390109, SantaCruz) at 4° C. overnight. The sections were washed with PBS three times, incubated with HRP conjugated anti-rat secondary antibody Sigma-Catalogue #AP136P) for F4/80 and anti-mouse immunoglobulins (Sigma Cat #AP130P) for MPO for 1 hour followed by washing 3 times with 1×PBS. Then, sections were incubated with stable diaminobenzidine (DAB, Invitrogen Catalog number: 750118) for 2-3-minutes to obtain clean staining. Sections were then washed with PBS and counterstained with hematoxylin. After dehydration with a series of increasingly concentrated ethanol washes, sections were mounted with neutral gum. Random fields at 10× magnification images were captured by a researcher blinded to the treatment using a Nikon microscope equipped with a computer (FIG. 16 and FIG. 19). The positive staining area and the total tissue area of each section were quantified using a Bioquant image analysis software program (Nashville, Tenn.). Results of positive staining were expressed as % of tissue area for each IHC staining (FIG. 17, FIG. 18, FIG. 20, and FIG. 21).

Results are expressed as mean±SEM/group. An unpaired T-test was used for statistical significance. Results are considered significant when p<0.05.

Results. Pre-treatment gross morphological observations. Three days after TNBS (or sham) infusion, one group of mice (n=5) was sacrificed to evaluate TNBS-induced effects on overall animal health and the colon. Sham-injected animals (n=2) were used as controls. As shown in FIG. 4, TNBS was found to reduce body weight (FIG. 4A), reduce colon length (FIG. 4B, FIG. 4D) and induce colon damage, as determined by colon damage score (FIG. 4C). The disease severity score DAI was determined as described in the literature (PPAR Res. 2018: 6079101). Briefly, a score of 0 (normal)-4 (severe) is assigned by two independent observers based on the gross morphological observations of body weight loss, colon appearance, stool consistency and rectal bleeding.

Overall, three days after TNBS administration, and prior to randomization of animals to vehicle or Compound 1 treatment groups, significant TNBS-induced damage to the colon was observed.

Colon Gross Morphological Endpoints. Animals were treated for 4 days with vehicle or Compound 1 (5 mg/kg, 15 mg/kg, and 45 mg/kg, BID, PO) and then animals were sacrificed. Animals were weighed, colons were collected and colon weight and length (FIG. 5, FIG. 6, FIG. 7, and FIG. 8). Overall disease severity was scored, as described previously (FIG. 9). Compound 1 treatment at 15 mg/kg and 45 mg/kg, BID, significantly restored body weight, colon weight and colon length. At 15 mg/kg and 45 mg/kg, there was also a marked improvement in Disease Severity Index.

Colon Histology. H&E stained colonic tissue sections (representative sections shown in FIG. 10 were scored by a two independent observers using a previously published system (Lab Invest. 1993 August; 69(2):238-49) for the following parameters: colon architecture (normal, 0—severe crypt distortion with loss of entire crypts, 3), degree of inflammatory cell infiltration (normal, 0—dense inflammatory infiltrate, 3), muscle thickening (normal, 0—marked muscle thickening present, 3), crypt damage/and goblet cells loss (0-3). Histological damage score of each individual score is shown in FIG. 11. The composite/total histological damage score is the sum of each individual score and is shown in FIG. 12. Histological scoring showed that Compound 1 treatment at 15 mg/kg and 45 mg/kg markedly improved colon histology compared to pre-treatment and vehicle treated animals.

Crypt Architecture/Goblet Cells. Colonic tissue sections were stained with Alcian Blue, to evaluate crypt architecture/goblet cells (representative sections shown in FIG. 13). The degree of Alcian Blue staining was quantified to determine crypt damage and goblet cell loss (FIG. 14 and FIG. 15). Compound 1 treatment at 15 mg/kg and 45 mg/kg significantly reduced TNBS-induced goblet cell loss compared to pre-treatment and vehicle treated animals (FIG. 14 and FIG. 15).

Cell infiltration markers myeloperoxidase (MPO) and F4/80. Myeloperoxidase (MPO) is abundantly expressed in neutrophils and F4/80 expression is restricted to eosinophils. Staining for these markers therefore is indicative of inflammation. Colon tissue was stained and intensity quantified for both MPO (representative sections shown in FIG. 16; quantitation in FIG. 17 and FIG. 18) and F4/80 (representative sections shown in FIG. 19; quantitation in FIG. 20 and FIG. 21). Quantitative analysis of MPO-stained colon tissue (FIG. 17 and FIG. 18 Error! Reference source not found.) showed that Compound 1 treatment at 5 mg/kg and higher significantly decreased MPO-staining versus TNBS+vehicle treated animals. Quantitative analysis of F4/80-stained colon tissue showed that test compound treatment at 15 mg/kg and higher significantly decreased F4/80-staining versus TNBS+vehicle treated animals (FIGS. 20 and 21).

### Example 4

### Inflammatory Bowel Disease Model: Dextran Sulfate Sodium (DSS)

DSS is a sulfated polymer that alters tight junction proteins, leading to the disruption of the intestinal barrier, and is toxic to epithelial cells. Results obtained in DSS-induced animal IBD models have helped to understand the pathogenesis of IBD and to screen potential therapeutic agents. DSS-induced IBD is simple and reproducible and results in symptoms resembling those of ulcerative colitis. The development of acute, chronic, or relapsing symptoms of IBD is dose-dependent, and the molecular weight of the DSS is key not only in the induction of colitis but also in the location of induction. For example, a comparative study found that colitis developed in animals treated with 5 kDa and 40 kDa DSS but not 500 kDa, and severe colitis developed in the lower colon following administration of 40 kDa, whereas mild colitis developed in the cecum and upper colon after 5 kDa. Various DSS dosage and duration are used to induce IBD in some animal models. Following DSS administration, animals usually develop colitis with weight loss and severe, bloody diarrhea. DSS colitis is characterized by mucosal ulceration, leukocyte infiltration, intestinal crypt distortion and epithelial hyperplasia.

It is believed that DSS colitis results in epithelial cell injury and increases the permeability of the intestinal mucosa to large molecules. DSS colitis is accompanied with dysregulation of the gut microbiota, and is associated with stimulation of innate and adaptive lymphoid elements and secretion of proinflammatory cytokines and chemokines. The percentages of CD3+T lymphocytes in Peyer's patches, natural killer (NK) and B lymphocytes in mesenteric lymph nodes, and NK CD8⁻ cells in intraepithelial lymphocytes are elevated in DSS-treated animals. Expression of P-selectin glycoprotein ligand-1, leukocyte function-associated antigen-1, and C—C chemokine receptor type 9 by T helper and cytotoxic T cells also increase after DSS treatment. Tissue cytokine and chemokine levels, including interleukin (IL)-1, IL-1, IL-6, IL-17, granulocyte colony-stimulating factor, granulocyte-macrophage colony-stimulating factor, eotaxin-1, monocyte chemoattractant protein 1, macrophage inflammatory protein (MIP)-1, and MIP-1 also change following exposure to DSS. Redox status is also disturbed as shown by decreased GSH and catalase (CAT) and increased reactive oxygen species (ROS), malondialdehyde (MDA), nitric oxide and myeloperoxidase (MPO). Activation of the nuclear factor-κB (NF-κB) pathway has been linked with the pathogenesis of DSS-induced colitis, and DSS induces cell death signaling by modulating B-cell lymphoma (Bcl)-2 and Bcl-2-associated X protein (Bax) apoptosis factors, and receptor-interacting protein 3, mixed lineage kinase domain-like protein, and caspase-8 necroptosis factors. Intestinal inflammation resulting from increased cell death has been reported to occur in IBD.

Adult male CD-1 mice were fed 3% DSS in drinking water for 5 days with alternate cycles of normal drinking water for 5 days and continued a total of 3 DSS cycles in 4 weeks. This model has been established to induce chronic colitis/IBD. Vehicle (n=10/group) or Compound 1 (50 mg/kg, PO, BID) were administered 72 hours after DSS and continued treatment for 4 weeks, then all mice were sacrificed. A small group (n=6) with normal drinking water was included as a sham control. Gross morphological and histopathological end points were evaluated. Additionally, tissue from the middle colon (100 mg) from all groups was subjected to hydroxyproline assay for collagen estimation as a fibrotic end point.

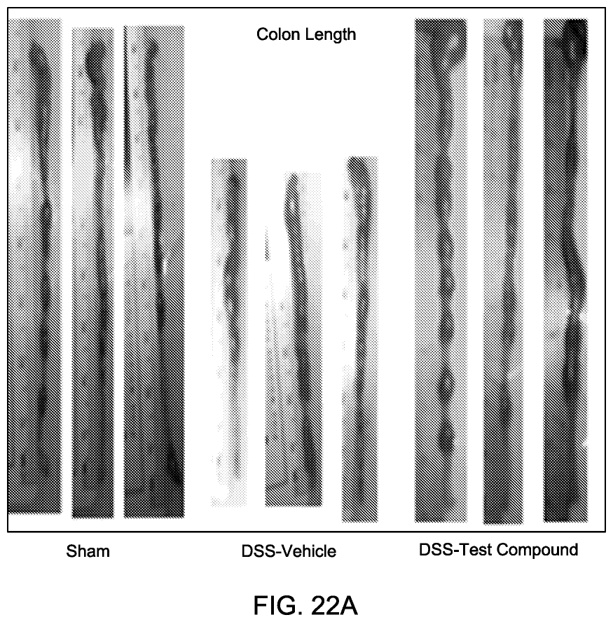

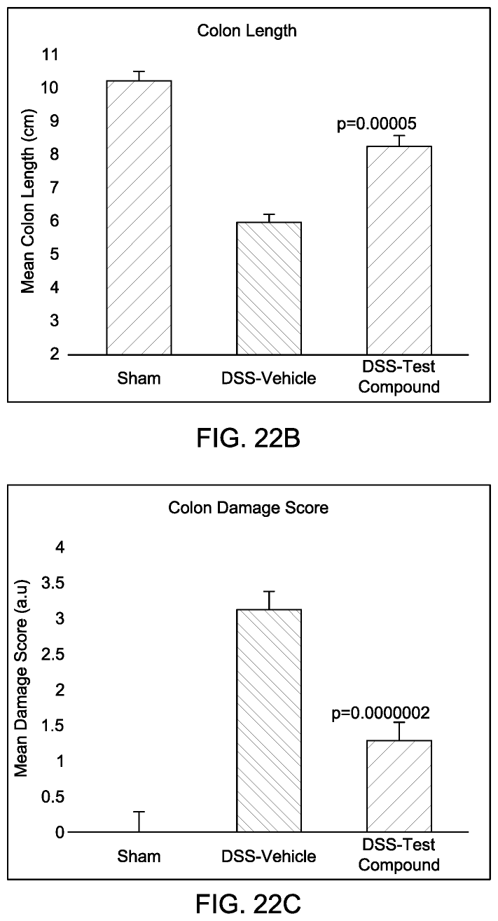

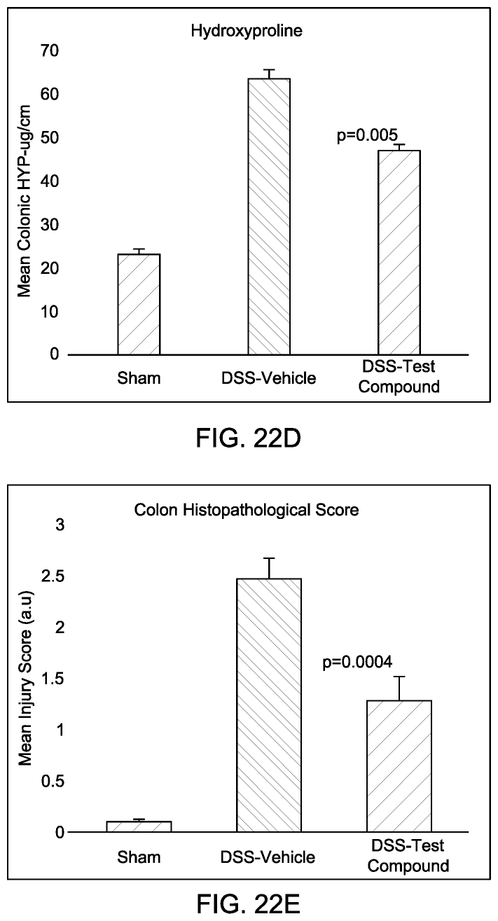

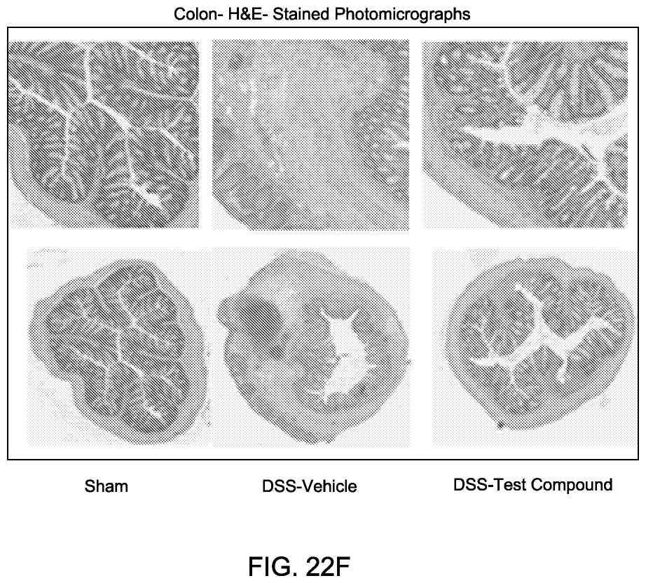

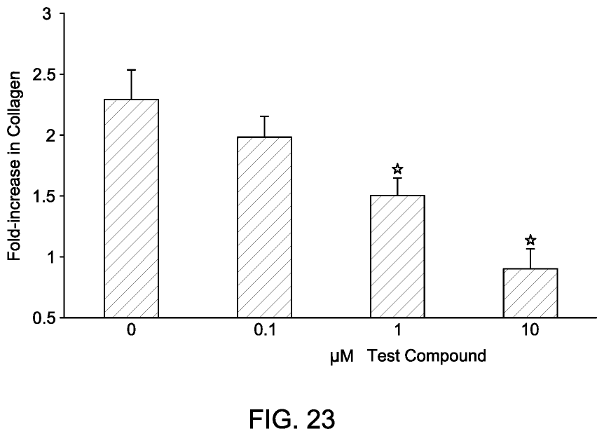

DSS-Vehicle cohort had the shortest colon length; DSS-Compound 1 cohort had significantly increased colon length compared to the DSS-vehicle cohort (FIG. 22A and FIG. 22B). Compound 1 treatment decreased macroscopic colon damage score in terms of appearance, inflammation and rectal bleeding, decreased colonic hydroxyproline (FIG. 22D) and histopathological injury score based on crypt damage, epithelial erosion and inflammatory cell infiltration as seen in H&E stained colon sections (FIG. 22C, FIG. 22E and FIG. 22F). These results indicate that Compound 1 could prevent progression of inflammation-induced fibrosis.

### Example 5

### Inflammatory Bowel Disease Model: 2,4,6-Trinitrobenzenesulfonic Acid (TNBS)

Using the same TNBS mouse model for IBD as described in Example 3, an experiment was performed with a 50 mg/kg dose of Compound 1, as described below.

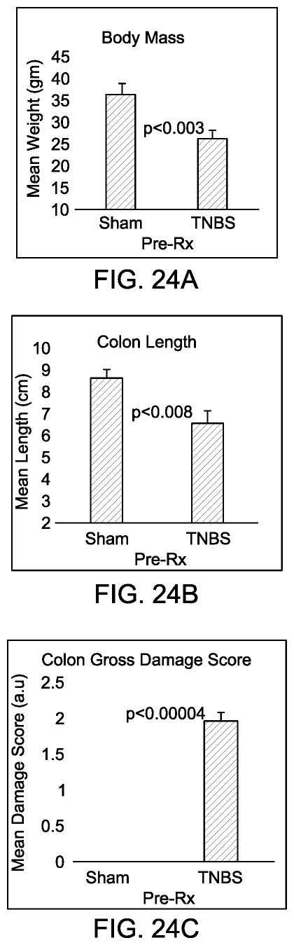

Male CD-1 mice were infused with TNBS (2 mg/mouse) in 45% ethanol and 5% PBS in 150 μL volume through rectal enema under isoflurane anesthesia. A sham group (n=6) received an equal volume of 45% ethanol and PBS mixture. At 72 hours following TNBS (or sham) infusion, a few mice were sacrificed to demonstrate the significant TNBS-induced IBD pathology including decreased body weight (FIG. 24A), reduced colon length (FIG. 24B), and increased colon damage (FIG. 24C).

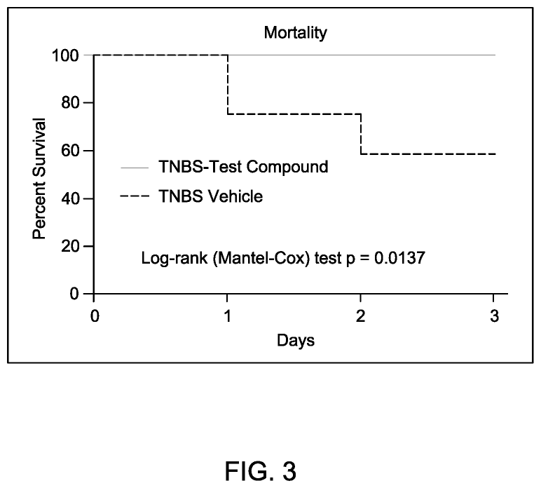

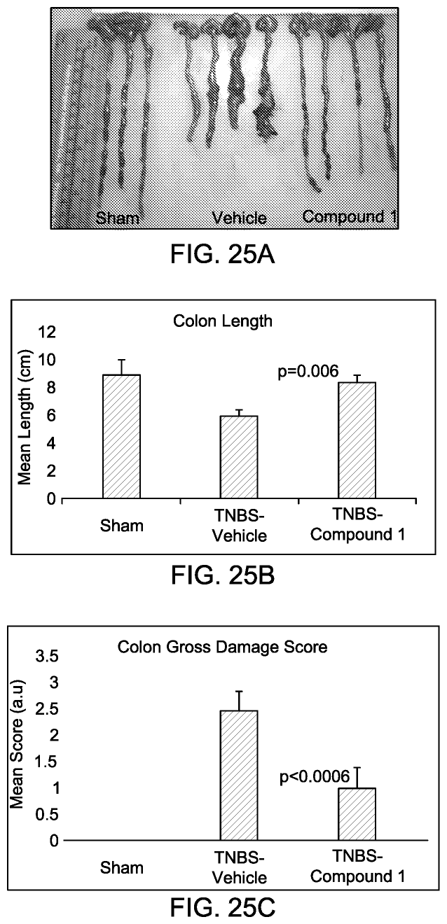

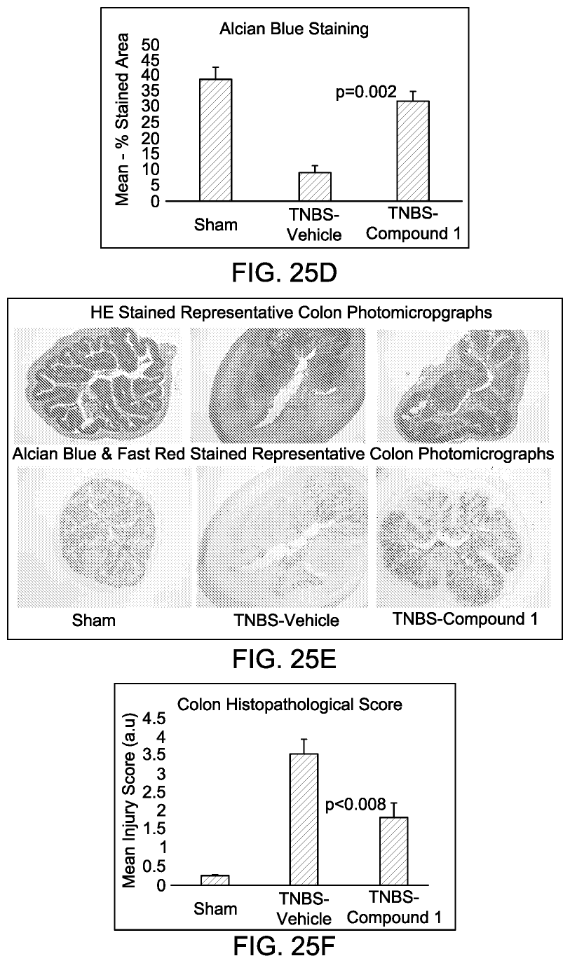

Remaining TNBS mice (n=12/group) were randomized to vehicle or Compound 1 at 50 mg/kg, PO, BID for 3 days. Delayed treatment with Compound 1 significantly decreased mortality (FIG. 3), restored colon length (FIG. 25A and FIG. 25B), decreased colon damage score (FIG. 25C), preserved colon architecture represented by Alcian blue staining for goblet cells of crypt/villi (FIG. 25D and FIG. 25E), and decreased colon histopathological score (FIG. 25F) compared to vehicle cohort.

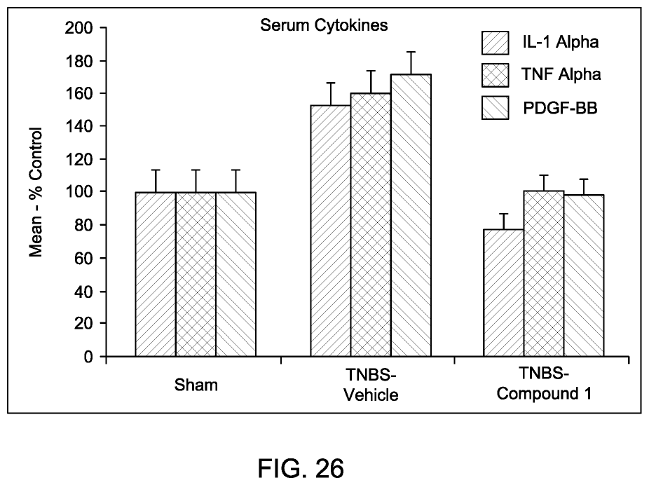

Consistent with observations in IBD patients, serum levels of interleukin-1α, TNFα and PDGF were elevated in the TNBS-induced colitis model. Compound 1 treatment significantly blocked the TNBS-induced increase in serum cytokines (FIG. 26).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for treating inflammatory bowel disease comprising administering to a subject in need thereof a compound of Formula (I): 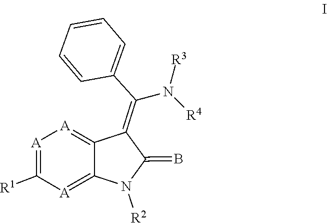
or a pharmaceutically acceptable salt thereof, wherein:
R¹ is —COOR⁵;
R² is H;
R³ and R⁴ are independently H, aryl, or heteroaryl, which may optionally be independently substituted with one or more lower alkyl, halogen, OR⁶, NO₂, CN, NH₂, NR⁶R⁷, NR⁶COR⁷ or NR⁶SO₂R⁷;
R⁵ is a lower alkyl group;
R⁶ and R⁷ are independently hydrogen, alkyl, cycloalkyl, heterocyclyl, cycloalkylalkyl, alkylcycloalkylalkyl, heterocycloalkyl or alkylheterocycloalkyl, which may be optionally substituted with alkyl, OR⁸, COOR⁸, NR⁸R⁹, or NHCOR⁸;
R⁸ and R⁹ are independently H or a lower alkyl group;
A is N or CH, wherein one A is nitrogen; and
B is O or S.

2. The method of claim 1, wherein the compound is of Formula (II): 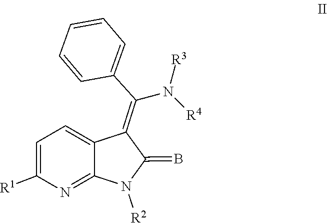
or a pharmaceutically acceptable salt thereof.

3. The method of claim 1, wherein the compound is of Formula (III): 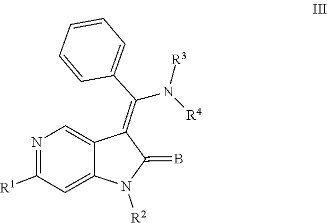
or a pharmaceutically acceptable salt thereof.

4. The method of claim 1, wherein the compound is of Formula (IV): 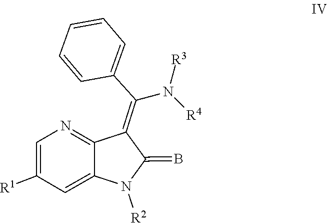
or a pharmaceutically acceptable salt thereof.

5. The method of any one of claims 1-4, wherein R² is hydrogen.

6. The method of any one of claims 1-5, wherein R³ is hydrogen.

7. The method of any one of claims 1-6, wherein R⁴ is phenyl optionally substituted with one or more lower alkyl, halogen, OR⁶, NO₂, CN, NH₂, NR⁶R⁷, NR⁶COR⁷ or NR⁶SO₂R⁷.

8. The method of claim 7, wherein R⁴ is phenyl substituted with NR⁶COR⁷.

9. The method of any one of claims 1-8, wherein R⁵ is methyl.

10. The method of any one of claims 1-9, wherein R⁶ is methyl.

11. The method of any one of claims 1-10, wherein R⁷ is

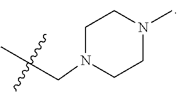 [SMILES: *CCN1CCN(C)CC1]

12. The method of any one of claims 1-11, wherein B is O.

13. The method of claim 1, wherein the compound is (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[2,3-b]pyridine-6-carboxylate, or a pharmaceutically acceptable salt thereof.

14. The method of claim 1, wherein the compound is (Z)-methyl 3-(((4-((3-(dimethylamino)-3-oxopropyl)(methyl)amino)phenyl)amino)(phenyl)methylene)-2-oxo-2,3-dihydro-1H-pyrrolo[2,3-b]pyridine-6-carboxylate, or a pharmaceutically acceptable salt thereof.

15. The method of claim 1, wherein the compound is (Z)-methyl 3-(((4-(2-(1,1-dioxidothiomorpholino)-N-methylacetamido)phenyl)amino)(phenyl)methylene)-2-oxo-2,3-dihydro-1H-pyrrolo[2,3-b]pyridine-6-carboxylate, or a pharmaceutically acceptable salt thereof.

16. The method of claim 1, wherein the compound is (Z)-methyl 3-(((4-(2-(dimethylamino)-N-methylacetamido)phenyl)amino)(phenyl)methylene)-2-oxo-2,3-dihydro-1H-pyrrolo[2,3-b]pyridine-6-carboxylate, or a pharmaceutically acceptable salt thereof.

17. The method of claim 1, wherein the compound is (Z)-methyl 3-(((4-(N-methyl-2-(4-methylpiperazin-1-yl)acetamido)phenyl)amino)(phenyl)methylene)-2-oxo-2,3-dihydro-1H-pyrrolo[2,3-b]pyridine-6-carboxylate, or a pharmaceutically acceptable salt thereof.

18. The method of claim 1, wherein the compound is (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[2,3-b]pyridine-6-carboxylate, or a pharmaceutically acceptable salt thereof.

19. The method of claim 1, wherein the compound is (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-c]pyridine-6-carboxylate, or a pharmaceutically acceptable salt thereof.

20. The method of claim 1, wherein the compound is (Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-c]pyridine-6-carboxylate, or a pharmaceutically acceptable salt thereof.

21. The method of claim 1, wherein the compound is (Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-c]pyridine-6-carboxylate, or a pharmaceutically acceptable salt thereof.

22. The method of claim 1, wherein the compound is (Z)-methyl 3-(((4-(2-(dimethylamino)-N-methylacetamido)phenyl)amino)(phenyl)methylene)-2-oxo-2,3-dihydro-1H-pyrrolo[3,2-c]pyridine-6-carboxylate, or a pharmaceutically acceptable salt thereof.

23. The method of claim 1, wherein the compound is (Z)-methyl 3-(((4-(N-methyl-2-(4-methylpiperazin-1-yl)acetamido)phenyl)amino)(phenyl)methylene)-2-oxo-2,3-dihydro-TH-pyrrolo[3,2-c]pyridine-6-carboxylate, or a pharmaceutically acceptable salt thereof.

24. The method of claim 1, wherein the compound is (Z)-methyl 3-(((4-(methyl(2-(4-methylpiperazin-1-yl)ethyl)amino)phenyl)amino)(phenyl)methylene)-2-oxo-2,3-dihydro-TH-pyrrolo[3,2-c]pyridine-6-carboxylate, or a pharmaceutically acceptable salt thereof.

25. The method of claim 1, wherein the compound is (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-b]pyridine-6-carboxylate, or a pharmaceutically acceptable salt thereof.

26. The method of claim 1, wherein the compound is (Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-b]pyridine-6-carboxylate, or a pharmaceutically acceptable salt thereof.

27. The method of claim 1, wherein the compound is (Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-b]pyridine-6-carboxylate, or a pharmaceutically acceptable salt thereof.

28. The method of claim 1, wherein the compound is (Z)-methyl 3-(((4-(2-(dimethylamino)-N-methylacetamido)phenyl)amino)(phenyl)methylene)-2-oxo-2,3-dihydro-1H-pyrrolo[3,2-b]pyridine-6-carboxylate, or a pharmaceutically acceptable salt thereof.

29. The method of claim 1, wherein the compound is (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-b]pyridine-6-carboxylate, or a pharmaceutically acceptable salt thereof.

30. The method of claim 1, wherein the compound is (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[3,2-b]pyridine-6-carboxylate, or a pharmaceutically acceptable salt thereof.

31. The method of any one of claims 1-30, wherein the subject is suffering from or susceptible to ulcerative colitis.

32. The method of any one of claims 1-31, wherein the subject is suffering from or susceptible to Crohn's disease.

33. The method of any one of claims 1-32, wherein the compound is administered as a pharmaceutical composition.
